(19) United States
(12) Patent Application Publication
(10) Pub. No.: US 2006/0127881 A1
(43) Pub. Date: Jun. 15, 2006

(54) Automated segmentation, classification, and tracking of cell nuclei in time-lapse microscopy

(75) Inventors: Stephen T.C. Wong, Boston, MA (US); Xiaowei Chen, Newton, MA (US)

Correspondence Address: CHOATE, HALL & STEWART LLP, TWO INTERNATIONAL PLACE, BOSTON, MA, 02110, US

(73) Assignee: Brigham and Women's Hospital

(21) Appl. No.: 11/257,523

(22) Filed: Oct. 25, 2005

Related U.S. Application Data
(60) Provisional application No. 60/621,856, filed on Oct. 25, 2004.

(51) Int. Cl. C12Q 1/00; C12Q 1/68; G06F 19/00; G06K 9/00

(52) U.S. Cl. 435/4; 435/6; 702/19; 382/128

(57) ABSTRACT

Methods and apparatus are provided for the automated analysis of images of living cells acquired by time-lapse microscopy. The new methods and apparatus can be used for the segmentation, classification and tracking of individual cells in a cell population, and for the extraction of biologically significant features from the cell images. Based upon certain extracted features, the inventive image analysis methods can characterize a cell as mitotic or interphase and/or can classify a cell into one of the following mitotic phases: prophase, metaphase, arrested metaphase, and anaphase with high accuracy.

### RELATED APPLICATIONS

The present application claims priority to Provisional Application No. 60/621,856 filed on Oct. 25, 2004 and entitled “Automated Segmentation, Classification, and Tracking of Cell Nuclei in Time-Lapse Microscopy”. The Provisional Application is incorporated herein by reference in its entirety.

### BACKGROUND OF THE INVENTION

Recent advances in imaging and microscopy technologies combined with the development of fluorescent probes that can be used in living cells allow cell biologists to quantitatively examine cell structures and functions at higher spatial and temporal resolutions than ever before. Time-lapse microscopy techniques (D. J. Stephens and V. J. Allan, Science, 2003, 300: 82-86) can provide a complete picture of complex cellular processes that occur in three dimensions over time. Information acquired by these methods allow dynamic phenomena such as cell growth, cell motion, cell nuclei division, metabolic transport, and signal transduction to be monitored and analyzed quantitatively.

Live-cell dynamic imaging techniques are also of great interest in the drug discovery and pharmacological research environments. Since most drugs are effective at the cellular level, drug screening can benefit from specific information about how drug candidates affect spatial and temporal events in whole living cells. High-content, high-throughput screening platforms based on time-lapse microscopy have been developed for performing cell-based assays; and these new screening tools are more and more frequently adopted by companies in the pharmaceutical and biotechnology industry.

High-resolution imaging of living cells offers significant advantages over fluorescence plate readers used in conventional cell-based assays. First, contrary to traditional approaches which assume that all cells under investigation are synchronized in their cell cycle and only measure cell populations' average response to a drug candidate, high-resolution imaging techniques can detect and record biological variability of individual cells within a population. In addition, high-resolution imaging screening enables simultaneous analysis of multiple target and/or pathway modulations by potential drug compounds. By providing a rich and diverse set of information about a drug candidate's effects on cellular processes, high-content, high-throughput imaging screening may facilitate the selection of drug candidates with higher probability of success in pre-clinical and clinical trials and thus reduce late stage failure rates of compounds in the pipeline.

Although time-lapse microscopy techniques can provide a large wealth of dynamic information regarding cell behavior, physiology, and morphology in the absence as well as in the presence of potential drug treatments, this information is currently far from being readily available. In fact, the analysis of live-cell images is still accomplished largely by time-consuming, labor-intensive manual methods, and most semi-automatic informatics tools for cell image analysis are extremely limited in their scope and capacity. In small scale studies, these manual and semi-automatic methods have yielded tremendous insights into the structures and functions of cellular constituents; however, these methods are unsuitable for the analysis of the staggering amounts of image data generated in high-content, high-throughput screening assays (P. D. Andrews et al., Traffic, 2002, 3: 29-36).

Automated systems are still lacking for the investigation of complex spatio-temporal cellular mechanisms such as cell-cycle behaviors. A clear understanding of the mechanism of cell cycle in the presence or absence of various perturbations can pave the way to the development of new therapeutic approaches for controlling or treating human diseases, such as cancer. Until recently, most studies of nuclear architecture were carried out in fixed cells (A. I. Lamond and W. C. Eamshaw, Science, 1998, 280: 547-553). However, time-lapse fluorescence microscopy imaging has since been demonstrated to allow live cell nuclei to be observed and studied in a dynamic fashion, and to provide far richer information content than conventional fixed-cell microscopy techniques (Y. Hiraoka and T. Haraguchi, Chromosome Res., 1996, 4: 173-176; T. Kanda et al., Curr. Biol., 1998, 8: 377-385). As shown in FIG. 1, cell cycle phases (e.g., interphase, prophase, metaphase, and anaphase) can be identified by measuring nucleus characteristics such as size, shape, location, concentration and/or amount. Therefore, automatic techniques to analyze cell cycle progress in living cells are of considerable interest for acquiring fundamental knowledge about the cell cycle of different cell types under various perturbation conditions as well as for the screening and discovery of new drugs that affect the cell cycle.

Clearly, the routine application of automated image analysis and large-scale screening is held back by substantial limitations in the tools currently used to store, process, and analyze the large volumes of information generated by time-lapse, live-cell microscopy. The potential of time-lapse microscopy techniques will not be fully realized until improved, automated, high-content analysis systems become available. In particular, systems that would allow biologists to track, analyze, and quantitate complex dynamic cellular mechanisms, such as cell-cycle behaviors, of individual cells in large cell populations are highly desirable.

### SUMMARY OF THE INVENTION

The present invention provides a new, powerful class of informatics tools for efficient dynamic cell imaging studies. More specifically, improved systems and strategies are described herein that can be used to quantitatively analyze complex spatio-temporal processes in individual cells. In particular, the present invention provides processes and apparatus with increased capacity to identify and track cell components and to extract biologically relevant cell components' features from large numbers of images acquired by time-lapse, live-cell microscopy. Furthermore, through selection and analysis of certain extracted features, the processes and apparatus of the present invention can automatically draw conclusions regarding certain aspects of the biology of a cell and can update these conclusions as the biology of the cell changes over time.

In certain embodiments, the methods and apparatus of the present invention allow for improved segmentation, classification, and tracking of individual cell nuclei in a cell population. The methods and apparatus of the present invention can also characterize a cell as mitotic or interphase and can further classify a cell into one of the following mitotic phases: prophase, metaphase, and anaphase.

More specifically, in one aspect, the present invention provides improved processes for the segmentation of cell components such as a cell's nucleus. Segmentation methods of the present invention comprise steps of: receiving a cell image showing the nucleus of one or more cells; performing a global threshold analysis of the cell image to generate a binary image; applying a watershed algorithm to segment any touching nuclei present in the binary image; and merging fragments of any over-segmented nuclei generated by the watershed algorithm using a shape and size merging process.

In certain embodiments of the invention, the one or more cells have been treated with a chemical or biological agent that selectively associates with a cell's nucleus (or a nuclear component such as nuclear DNA or nuclear proteins). Preferably, the agent emits a signal whose intensity is proportional to the amount of nuclear component to which it is associated.

Global threshold analysis according to the present invention may be carried out using any suitable algorithm. For example, performing a global threshold analysis may comprise using an isodata algorithm.

In certain embodiments, the shape and size-based merging process is an iterative process which finds the smallest touching objects at each iteration; calculates the size of the smallest nucleus in the image; and merges these touching objects based on considerations regarding their size and, optionally, their compactness.

More specifically, in certain embodiments, the shape and size merging process comprises steps of: measuring the size, T_{size}, of the smallest nucleus in the cell image; identifying a first fragment touching a second fragment, wherein the second fragment is the smallest fragment touching the first fragment; if the size of the first fragment is lower than T_{size}, merging the first and second fragments; if the size of the first fragment is greater than T_{size}, calculating the compactness of the first fragment, the compactness of the second fragment and the compactness of an object consisting of the first fragment merged with the second fragment; and if the compactness of the object is lower than the compactness of the first fragment or of the second fragment, merging the first and second fragments.

In another aspect, the present invention provides methods for the characterization of a cell nucleus. In certain embodiments, methods of the invention comprise steps of: receiving a cell image showing the nucleus of one or more cells; performing a segmentation analysis of the cell image to obtain a segmented digital image; and extracting one or more parameters from the segmented digital image to characterize the nucleus of at least one of the cells of the cell image.

In some embodiments, the segmentation analysis is performed by the new methods disclosed herein. The segmentation analysis provided a segmented digital image that comprises a representation of the nucleus of each of the one or more cells, each representation comprising a collection of signal intensity values at positions in the image where the nuclear component is present.

Extracting one or more parameters from the segmented digital image to characterize the nucleus of at least one cell comprises extracting from the representation of each nucleus to be characterized a feature selected from the group consisting of maximum of grey levels, minimum of grey levels, average of grey levels, standard deviation of grey levels, length of nucleus major axis, length of nucleus minor axis, nucleus elongation, nucleus area, nucleus perimeter, nucleus compactness, nucleus convex perimeter, nucleus roughness, and combinations thereof.

In still another aspect, the present invention provides processes allowing for improved tracking of cell components in space and time. In particular, using the inventive processes, it is possible to track nuclei during cell mitosis and division. In certain embodiments, processes are provided that comprise steps of: obtaining a sequence of images showing the nucleus of one or more cells, wherein the images are recorded at consecutive time points and each image is associated with a specific time point; performing a segmentation analysis of each image of the sequence to obtain a sequence of segmented digital images, wherein each segmented digital image is associated with the time point of the cell image from which it is obtained; performing a correction of any frame shift in the segmented digital images; and applying a matching algorithm to find, for each nucleus in a first segmented image of the sequence, possible matching nuclei in a second segmented image of the sequence, wherein the second image is consecutive to the first image.

In some embodiments, applying a matching algorithm comprises using an iterative algorithm in which nuclei in two consecutive frames of the image sequence are considered at each iteration. Preferably, the algorithm finds, for each nucleus in a first image frame, possible matching nuclei in the following image frame, by computing the distance between them. More specifically, applying a matching algorithm to find possible matching nuclei in a second image for each nucleus in a first image may comprise steps of: calculating, for each nucleus in the first image, the distance between the nucleus and a possible matching nucleus in the second image; and determining that the nucleus in the second image matches the nucleus in the first image if the distance calculated is below a pre-determined threshold.

In certain embodiments, the tracking method further comprises solving any ambiguous correspondences generated by the matching algorithm. Solving any ambiguous correspondences may comprise identifying any false ambiguous correspondences; and applying a size and location-based tracking algorithm to solve the remaining ambiguous correspondences. The size and location-based tracking algorithm solves ambiguous correspondences by comparing the size and/or location of matching nuclei over more than two image frames. In some embodiments, applying a size and location-based tracking algorithm comprises calculating one or more of nucleus size, nucleus size change from one image to another, nucleus location, nucleus location change from one image to another, relative size of two nuclei in an image, relative location of two nuclei in an image, relative size change of two nuclei from one image to another, relative location change of two nuclei from one image to another, nucleus center of gravity, distance between two centers of gravity, and combinations thereof.

In another aspect, the present invention provides methods for the identification of cell cycle states. These methods include steps of: receiving a cell image showing the nucleus of one or more cells; performing a segmentation analysis of the cell image to obtain a segmented digital image; extracting one or more parameters from the segmented digital image to characterize the nucleus of at least one of the cells of the cell image; and classifying the at least one cell into a cell cycle state based on the one or more extracted parameters.

In these methods, the segmented digital image may be obtained using one of the segmentation processes disclosed herein. Similarly, extracting one or more parameters from the segmented digital image to characterize the nucleus of at least one cell may be performed by extracting from the representation of each nucleus to be characterized a feature selected from the group consisting of maximum of grey levels, minimum of grey levels, average of grey levels, standard deviation of grey levels, length of nucleus major axis, length of nucleus minor axis, nucleus elongation, nucleus area, nucleus perimeter, nucleus compactness, nucleus convex perimeter, nucleus roughness, and combinations thereof, as described above.

In some embodiments, the step of classifying the cell into a cell cycle state based on the one or more parameters comprises selecting an optimal subset of features from the set of extracted features. The selection of a subset of features may be performed by any suitable method. An optimal subset of parameters may be selected by using a sequential forward selection method, wherein the discrimination power of the parameters is evaluated by a K-Nearest Neighbor classifier. The classifier may be optimized with training data.

In certain embodiments, the processes of the invention are used to classify individuals cells in a cell population as interphase or mitotic. In other embodiments, the processes of the invention are used to classify individual cells in a cell population into one of the following mitotic phases: prophase, metaphase, arrested metaphase, and anaphase.

According to the same aspect, the present invention provides improved processes for the identification of the cell cycle state of a cell over a period of time. These improved processes are similar to the methods already described above but further comprise tracking the nucleus of the cells whose cell cycle is under study and correcting any cell cycle identification errors suing biological knowledge-driven heuristic rules. Heuristic rules are preferably selected from the group consisting of the phase progression rule, the phase continuation rule, the phase timing rule, and any combination thereof.

In another aspect, the present invention provides methods for identifying or screening compounds or agents that have an effect (e.g., a perturbing or regulating effect) on cell cycle.

In another aspect, the present invention provides methods for diagnosing a disease or condition associated with cell cycle perturbation.

In another aspect, the present invention provides machine-readable media on which are provided program instructions for performing one or more of the inventive processes of image analysis. In still another aspect, the present invention provides computer products comprising a machine-readable medium on which are provided program instructions for performing one or more of the inventive processes. In yet another aspect, the present invention provides an image analysis apparatus comprising a memory adapted to store, at least temporarily, at least one image acquired by time-lapse microscopy, and a processor configured or designed to perform one or more of the inventive processes. In certain embodiments, the image analysis apparatus further comprises an interface adapted to receive one or more cell images and/or an image acquisition system that produces one or more cell images.

These and other objects, advantages and features of the present invention will become apparent to those of ordinary skill in the art having read the following detailed description.

### BRIEF DESCRIPTION OF THE DRAWING

FIG. 1 presents a series of pictures showing the appearance of a cell's nucleus in different phases of the cell cycle.

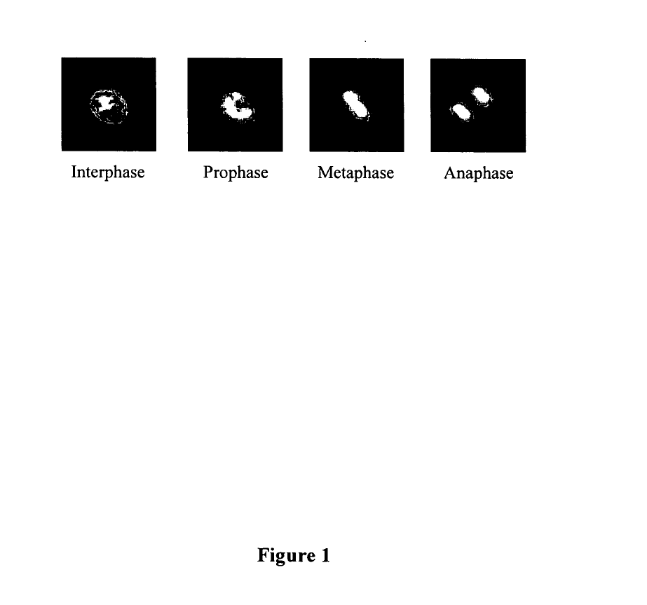

FIG. 2 is a process flow diagram depicting, at a high level, the system architecture of one embodiment of the inventive method of dynamic cellular image analysis.

FIG. 3 is a process flow diagram according to one embodiment of the segmentation process disclosed in the present invention.

FIG. 4 presents a set of pictures showing an example of thresholding/watershed segmentation according to the invention. The grey level image is presented in (A); the corresponding binary image obtained after applying the threshold is presented in (B); the distance map, which is linearly mapped to 0-255 for display purpose, is presented in (C); and the corresponding watershed segmentation is presented in (D).

FIG. 5 presents pictures showing two examples of nucleus fragments merging according to the invention. In FIG. 5(A), the two small over-segmented fragments are merged based on their size. In FIG. 5(B), two large fragments are merged based on consideration of their compactness.

FIG. 6 is a high level flow diagram in accordance with one embodiment of the inventive process of cell cycle phase identification.

FIG. 7 shows four different schemes (Cases A to D) used in the text to illustrate how to apply knowledge-driven heuristic rules to correct cell phase identification errors according to the present invention. Schemes A-D show portions of cell sequences, wherein 1 stands for interphase, 2 for prophase, 3 for metaphase, 4 for anaphase, and 5 for arrested metaphase, and wherein bold font marks the places where the errors happened.

FIG. 8 presents a set of pictures showing nuclei/DNA migration during division. Nuclei/DNA are shown before division in (A) and (C) and after division in (B) and (D), respectively.

FIG. 9 presents a series of consecutive image subframes from a time-lapse sequence showing the changes in nucleus/DNA appearance during cell mitosis (A)-(H).

FIG. 10 shows a high level process flow diagram in accordance with one embodiment of the inventive tracking method.

FIG. 11 presents three series of pictures showing different examples of nucleus divisions: in (A), a single nucleus division; in (B), a multiple nuclei division; and in (C), a single nucleus dividing into more than two daughter cell nuclei.

FIG. 12 shows a scheme depicting the two possible cases of ambiguous correspondences: in case (a) a one-to-many correspondence, and in case (b) a many-to-one correspondence.

FIG. 13 shows two schemes depicting examples of ambiguous correspondence caused by under-segmentation. In (a), the ambiguous correspondence is due to nuclei touching (at time t+1), and in (b), the ambiguous correspondence is due to nuclei overlapping (at time t+1).

FIG. 14 presents a set of pictures showing examples of ambiguous correspondence caused by nucleus division. In (A), the nuclei are shown before division, in (B) the nuclei are shown after division.

FIG. 15 presents a set of pictures showing the results obtained using different watershed segmentation methods in four different cases. Part (A) shows portions of the original gray level images. Part (B) shows the binary images obtained after isodata thresholding. Part (C) shows the results obtained using the watershed segmentation method. Part (D) shows the results obtained using the method of watershed segmentation with connectivity-based merging, and part (E) shows the results obtained with the inventive segmentation method (i.e., watershed segmentation with shape and size-based merging).

FIG. 16 is a graph showing the variation of the performance of the inventive classifier (the performance being defined as the ratio between the number of nuclei correctly identified and the total number of nuclei) as a function of the size of the feature subset.

### DETAILED DESCRIPTION OF CERTAIN PREFERRED EMBODIMENTS

Improved systems and strategies for dynamic cell image analysis are described herein. More specifically, the present invention relates to processes (methods) and apparatus with increased capacity to identify and track objects in time and space, and to extract, analyze and quantitate object features from large amounts of images acquired using time-lapse, live-cell microscopy. Furthermore, based on certain extracted features, the inventive processes and apparatus can automatically draw conclusions about certain aspects of the biology of a cell. The present invention also relates to machine-readable media on which are provided program instructions, data structures, etc, for performing one or more of the inventive processes.

In particular, the methods and apparatus of the present invention allow for improved segmentation, classification, and tracking of individual cell nuclei in a cell population. Through extraction, selection and analysis of biologically significant nuclei features, the processes and apparatus of the present invention can characterize a cell as mitotic or interphase, and can further classify a cell into one of the following mitotic phases: prophase, metaphase, and anaphase.

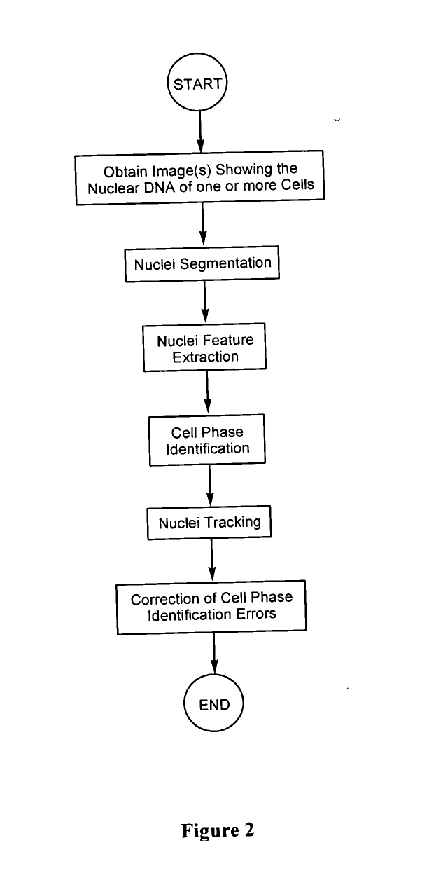

A high level process flow diagram in accordance with one embodiment of the present invention is depicted in FIG. 2. Each step or module of the inventive process is described in detail below.

### I—Dynamic Cell Images

As shown in FIG. 2, the inventive image analysis process generally starts where one or more image analysis tools (typically logic implemented in hardware and/or software) obtain one or more live cell images showing the nucleus of at least one cell. In certain embodiments, a single cell image is obtain at the beginning of the image analysis process. In other embodiments, a series (or sequence) of images acquired over time for a given cell or cell population (i.e., two cells or more) is obtained. In the latter case, the images are recorded at consecutive time points and each image of the sequence is associated with a specific time point.

The one or more images provided at the start of the inventive process are recorded by an image acquisition system, such as a time-lapse microscopy instrument. In one embodiment, the image acquisition system is directly coupled with the image analysis tool of the present invention. Alternatively, the one or more images under consideration may be provided by a remote system unaffiliated with the image acquisition system. For example, the images may be acquired by a remote image analysis tool and stored in a database or other repository until they are ready to be analyzed by the image analysis processes/apparatus of this invention.

Images may be taken from an assay plate or other cell support mechanism in which multiple cells are growing or stored. Preferably, cells that are imaged are live cells. The terms “live cell” and “living cell” are used herein interchangeably. They refer to a cell which is considered living according to standard criteria for that particular type of cell, such as maintenance of normal membrane potential, energy metabolism, or proliferative capability.

Cells may be any of a variety of normal and transformed cells that can be grown in standard tissue culture ware. Preferably, cells are of mammalian (human or animal) origin. Mammalian cells may be of any organ or tissue origin (e.g., brain, liver, lung, heart, etc) and of any cell types (e.g., basal cells, epithelial cells, platelets, lymphocytes, T-cells, B-cells, natural killer cells, macrophages, tumor cells, etc). Cells may be primary cells, secondary cells or immortalized cells (i.e., established cell lines). They may have been prepared by techniques well known in the art (for example, cells may be obtained by drawing blood from a patient or healthy donor or they may be isolated from a tissue obtained from a patient or healthy donor by biopsy) or they may have been purchased from immunological and microbiological commercial resources (for example, from the American Type Culture Collection, Manassas, Va.). Alternatively or additionally, cells may have been genetically engineered to contain, for example, a gene of interest such as a gene expressing a growth factor or a receptor.

Generally, the images used as the starting point for the analysis methods of the present invention are obtained from cells that have been specifically treated and/or imaged under conditions that contrast markers of cellular components of interest from other cellular components and from the background of the image. Preferably, the cells are specifically treated and/or imaged under conditions that contrast the cells' nuclei from other cellular components and the background of the image. For example, images may be obtained of cells that have been treated with a chemical or biological agent that specifically renders visible (or otherwise detectable in a region of the electromagnetic spectrum) the nucleus of the cells. In certain embodiments, cells have been treated with a chemical or biological agent that specifically renders visible a nuclear component. Nuclear components that can be rendered visible or detectable include, but are not limited to, nuclear DNA and nuclear proteins.

Common examples of chemical agents that can be used to render visible a cell's nucleus are colored dyes or fluorescent, phosphorescent or radioactive compounds that bind directly or indirectly (e.g., via antibodies or other intermediate binding agents) to the cells' nucleus, to specific sequences of DNA or to regions of a chromosome. In certain embodiments, the cells are treated with a fluorescent DNA staining agent.

Examples of such compounds include fluorescent DNA intercalators and fluorescently labeled antibodies to DNA or other nuclear components. Examples of fluorescent DNA intercalators include DAPI (i.e., 4′,6-diamidino-2-phenylindole, which shows blue fluorescence upon binding to DNA and can be excited with a mercury-arc lamp or with the UV lines of the argon-ion laser) and bisbenzimide dyes (such as Hoechst 33258, Hoechst 33342, Hoechst 34580 and Hoechst 33341, which are cell membrane-permeant, minor groove-binding DNA stains that fluoresce bright blue upon binding to DNA). These and other fluorescent DNA intercalators are commercially available, for example, from Molecular Probes, Inc. (Eugene, Oreg.).

Alternatively, the cells may have been treated with a biological agent that renders the cells' nuclei visible or detectable. For example, the cells may have been genetically engineered to express a gene encoding a fluorescent marker, such as the green fluorescent protein, GFP (or any of its derivatives). As a general rule, transgenic expression of GFP within any given cell requires simply placing the GFP coding sequence (or slightly modified versions of the sequence) under the transcriptional control of appropriate regulatory sequences. GFP has many characteristics that make it a particularly convenient marker for live cell imaging. GFP is a biomolecule derived from the pacific jellyfish Aequova aequora, which has been found to be non-toxic to cells of many organisms. Formation of the fluorescent chromophore occurs as an intramolecular reaction sequence that is limited only by the availability of molecular oxygen. This reaction is independent of cellular co-factors. GFP and its derivatives as well as methods for genetically engineering cells to express these biomolecules are well known in the art.

Preferably, the agent or marker is selected such that it generates a detectable signal whose intensity is related (e.g., proportional) to the amount of nuclear component (e.g., DNA) to which it is bound. Since the absolute magnitude of signal intensity can vary from image to image due to changes in the cell staining and/or image acquisition procedure and/or apparatus, a correction algorithm may be applied to correct the measured intensities. Such algorithms can easily be developed based on the known response of the optical system used under a given set of acquisition parameters.

### II—Dynamic Cellular Image Analysis Method

As shown in FIG. 2, one embodiment of the inventive method of dynamic cellular image analysis is designed to produce an accurate description of each nucleus in one or more images of an image sequence (the segmentation step); to extract nuclei descriptors (the feature extraction step); to identify cell phase (the classification step); and to keep track of the nuclei across the image sequence (the tracking step).

As will be apparent to those skilled in the art, the present invention may be practiced without using some of the specific details disclosed herein. Furthermore, some operations, modules, steps, or features may be omitted, and often alternative elements or processes may be substituted.

A—Cell Nuclei Segmentation

After an image showing the cells' nucleus has been obtained, the image is segmented into discrete images/representations of the nucleus in each cell. Segmentation generates a “nuclei mask” that can then be used to perform image analysis on a cell-by-cell basis.

Preferably, each image/representation of the nucleus in a cell is limited to those pixels where the nucleus (or nuclear component, e.g., DNA) of the cell is present. Each of these pixels (i.e., positions in the image) is associated with a signal intensity value representing the amount of nucleus (or nuclear component) present at the corresponding location. The shape of each image/representation corresponds to the boundaries within which the nucleus (or nuclear component, e.g., DNA) lies. It is worth noting here that, generally, in interphase mammalian cells, the DNA is contained entirely within the cell's nucleus, while in mitotic cells, the DNA does not reside within a nucleus.

Segmentation is an important part of an automated cellular analysis system, as the results of the segmentation process directly affect the accuracy of the subsequent cell-cycle phase identification and cell-tracking.

Segmenting an object (such as a cell's nucleus) in a time-lapse microscopy image is a relatively easy task, usually implemented with thresholding, region growing or edge detection (see, for example, P. Ahrens et al., J. Microscopy, 1990, 157: 349-365; C. Garbay et al., Anal. Quant. Cytol. Histol., 1986, 8: 25-34; T. Kirubarajan et al., in “Multitarget-Multisensor Tracking: Applications and Advances”, Y. Bar-Shalom and W. D. Blair (Eds.), Artech House: Norwood, Mass., 2000, 3: 199-231; C. MacAulay and B. Palcic, Anal. Quant. Cytol. Histol., 1998, 10: 134-138; G. Wolf, Proc. SPIE, 1992, 1660: 397-408). Most of the algorithms used for segmentation take into account either morphological information or pixel information present in each image. Problems arise when trying to segment touching objects since in such a situation it is difficult to define the boundary of each object.

Watershed techniques can be used to segment touching objects (see, for example, A. Bleau and J. L. Leon, Computer Vision and Image Understanding, 2000, 77: 317-370; M. Norberto et al., Cytometry, 1997, 28: 289-297; P. S. Umesh Adiga and B. B. Chaudhuri, Pattern Recognition, 2001, 34: 1449-1458). However, watershed techniques often generates over-segmented fragments, and to deal with the over-segmentation problem, additional processing is needed to merge the fragments. To this end, Umesh Adiga and Chaudhuri (Pattern Recognition, 2001, 34: 1449-1458) have used a connectivity-based merging method wherein a tiny cell fragment is merged with a nearby cell if it shares the maximum boundary with that cell. They applied this method on a set of 327 cells and reported a 98% correct segmentation rate. This method, however, can only merge small cell fragments and would wrongly consider large fragments with a size above a certain preset value, as individual cells.

Bleau and Leon (Computer Vision and Image Understanding, 2000, 77: 317-370) have used an iterative try-and-test approach to merge small regions with their nearby larger regions based on a set of criteria related to volume, depth, and surface criteria. They applied this method to segment the vesicles in live cells but did not report any experimental results.

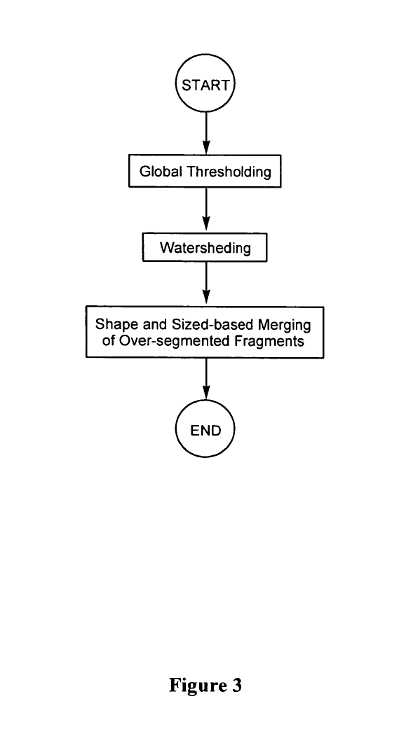

An improved segmentation process is provided herein. FIG. 3 shows a flow diagram in accordance with one embodiment of the inventive segmentation method. In short, in this process, the objects (e.g., cell nuclei) are first segmented out from the background by applying a global thresholding technique; touching objects are then separated by watersheding; and a shape and size-based method is used to merge over-segmented fragments. Each step of the inventive segmentation process is described in detail below.

Image Thresholding and Separation of Touching Nuclei

In time-lapse fluorescence microscopy images, nuclei are bright objects protruding out from a relatively uniform dark background. The segmentation process of the present invention starts by applying a thresholding technique. Thresholding is based on simple, well-known concepts. A parameter, called the brightness threshold is chosen and applied to each pixel of the image under consideration as follows: (1) if the intensity of the pixel is higher than the brightness threshold, the pixel is considered as belonging to an object, (2) if the intensity of the pixel is lower that the brightness threshold, the pixel is considered as belonging to the background.

Generally, the threshold value is chosen from the brightness histogram of all or part of the image that is being segmented. A variety of techniques/algorithms have been devised to automatically select a threshold value starting from the gray-value histogram. Many of these algorithms can benefit from a smoothing of the raw histogram data to remove small fluctuations. These smoothing algorithms include, but are not limited to, the background-symmetry algorithm, the triangle algorithm, and the isodata algorithm. In certain embodiments of the segmentation process of the present invention, thresholding comprises the use of an isodata algorithm (M. Norberto et al., Cytometry, 1997, 28: 289-297; N. Otsu, IEEE Trans. on System, Man and Cybernetics, 1978, 8: 62-66, each of which is incorporated herein by reference in its entirety).

The isodata algorithm is an iterative technique, wherein the brightness histogram is initially segmented into two parts using a starting threshold value, such as, for example, half the maximum dynamic range. The sample mean of the gray values associated with the foreground pixels and the sample mean of the gray values associated with the background pixels are computed; the average of these two sample means is then considered as the new threshold value. The process is repeated, based upon the new threshold, until the threshold value does not significantly change anymore.

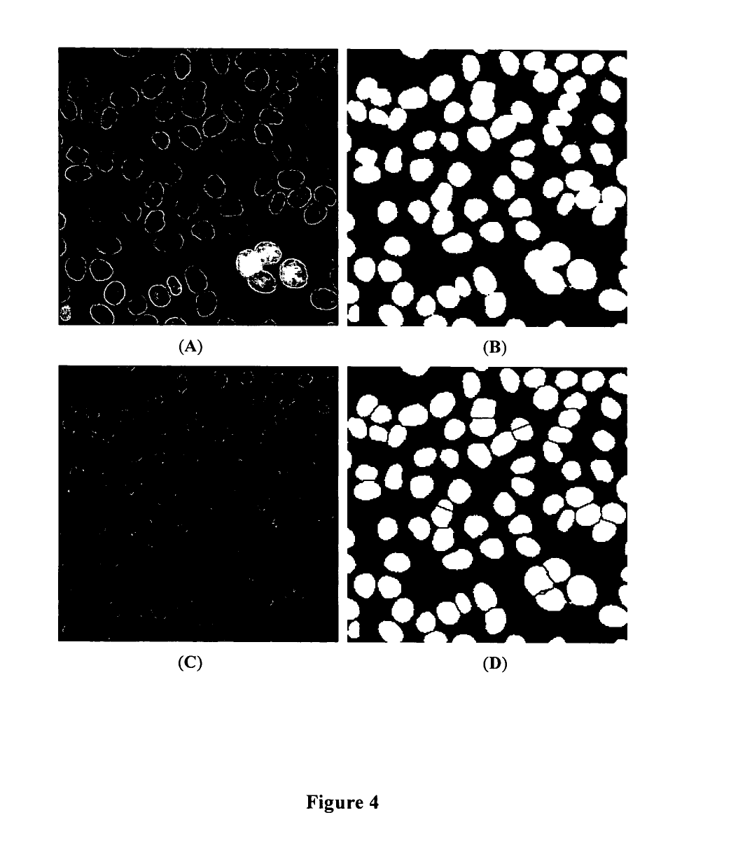

As shown on FIG. 4(B), this algorithm correctly segments most isolated nuclei, but is unable to segment touching nuclei. The algorithm fails because it classifies the pixels into only two distinct groups (object and background). If two nuclei are so close that there are no background pixels between them, the algorithm cannot separate them.

To deal with touching objects, the segmentation process of the present invention comprises the step of applying a watershed algorithm (S.-F. Chang et al., IEEE Trans. on Circuits and Systems for Video Technology, 1998, 8: 602-615, which is incorporated herein by reference in its entirety). The watershed algorithm first calculates the Euclidian distance map (EDM) of the binary image developed with the isodata algorithm. It then finds the ultimate eroded points (UEP), which are the local maxima LMax[l] of the Euclidian distance map. The watershed algorithm then dilates each of the ultimate eroded point as far as possible—either until the edge of the nucleus is reached or until the edge of the region of another ultimate eroded point is reached.

FIG. 4(C) shows an example of an Euclidian distance map calculated from the binary image presented on FIG. 4(B), and FIG. 4(D) shows the resulting segmentation.

Shape and Size-Based Fragments Merging

When there is more than one ultimate eroded point within the same object (nucleus), the watershed algorithm fails. In such a case, the object will be incorrectly divided into several fragments. Therefore, a merging processing is needed to correct such segmentation errors. As shown in the process flow diagram presented on FIG. 3, the segmentation process of the present invention uses a shape and size-based merging technique to merge over-segmented fragments.

On an image, nuclei usually appear as elliptical objects, which exhibit various degree of ellipticity. Factors that may be used to describe the ellipticity of each nucleus include compactness. Compactness is defined as the ratio of the square of the perimeter of the nucleus to the area of the nucleus, as show in the following equation:

$$Compactness = \frac{{Perimeter}^{2}}{4 π \times Area} \qquad ( 1 )$$

Compactness, which is equal to 1 when the shape of the imaged nucleus is a circle, increases as the nucleus contour becomes less circular and rougher. If a round nucleus is divided into several fragments, the compactness of each fragment will be larger than the compactness of the entire nucleus. The merging technique of the present invention takes into account this observation; and identifies over-segmented nucleus fragments based on their size and shape, and then merges them into single nucleus units.

The merging process itself can be described as follows. Let N be the total number of segmented objects found by the watershed segmentation algorithm. Let T_{size} be the size of the smallest nucleus in the image. The process evaluate all touching objects using a checking procedure. Two objects are considered touching if they belong to the same object in the binary image before the watershed algorithm was applied. The iterative merging process finds the smallest touching objects in each iteration, and then uses this checking process to update the segmentation until no more touching objects can be merged.

The checking process is implemented as follows: (1) if the size of a touching object is less than T_{size}, it is merged with the smallest touching neighbor; (2) if the size of a touching object is greater than T_{size}, three compactness values are calculated; namely, the compactness of the object, the compactness of the object's touching neighbor, and the compactness of the two objects as a whole (i.e., object and neighbor after merging). If the compactness of the two objects after merging is lower than the compactness of the object or its touching neighbor, then the two objects are merged.

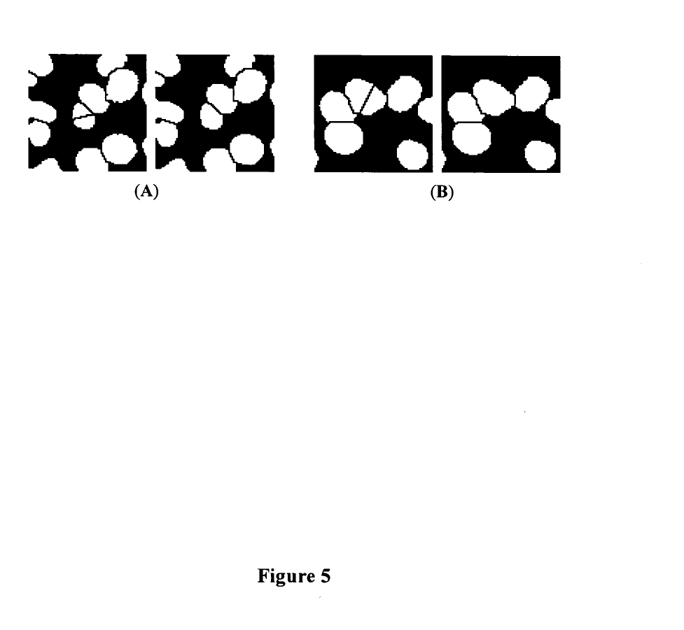

FIG. 5 shows two examples of merging performed using the inventive merging process. In FIG. 5(A), the two small over-segmented fragments are merged based on their size. In FIG. 5(B), two large fragments are merged based on compactness considerations.

After nuclei segmentation, a morphological closing process is generally performed on the resulting binary images in order to smooth the nuclei boundaries and fill holes inside objects (see, for example, S. Chen and M. Haralick, IEEE Trans. on Image Processing, 1995, 4: 335-345, which is incorporated herein by reference in its entirety). These binary images are then used as a mask on the original image to produce the final segmentation resulting in a segmented cell image. This segmented cell image can then be used for extraction of nuclei features.

Example 1 describes the results obtained using the inventive segmentation method in time-lapse microscopy images of live GFP-modified HeLa cells. The experimental results obtained using the inventive method showed an accuracy of 97.8% of nucleus segmentation. A comparison experiment demonstrated that the inventive shape and size-based merging technique could correctly merge 82.4% of the over-segmented nuclei fragments obtained after watersheding, while the connectivity-based merging technique used by Umesh Adiga and Chaudhuri (Pattern Recognition, 2001, 34: 1449-1458) could only merge 36.4% of them.

B—Feature Extraction and Cell Phase Identification

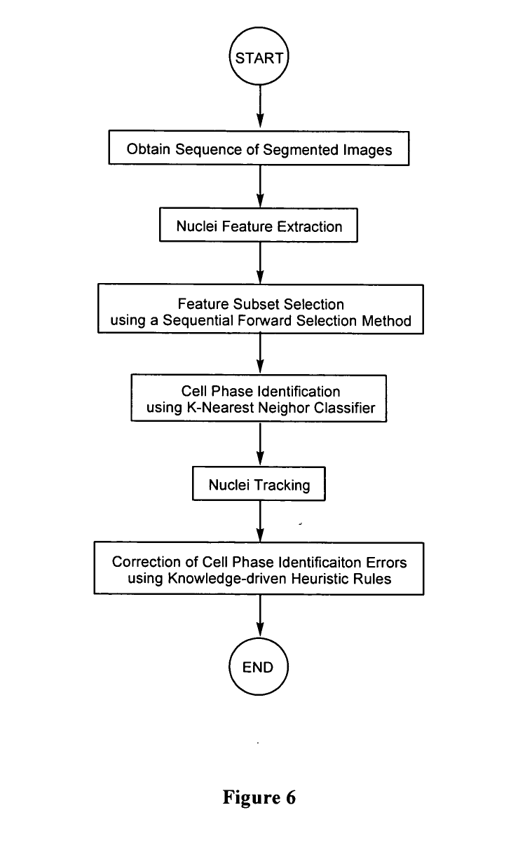

In another aspect, the present invention provides image analysis processes that allow for improved cell phase identification. FIG. 6 shows a high level process flow diagram in accordance with one embodiment of the inventive cell phase identification method.

The inventive segmentation process described above produces an accurate description (image/representation) of each object (e.g., cell nucleus) of an image sequence acquired by time-lapse, live-cell microscopy. As already mentioned, each image/representation is a collection of signal intensity values as a function of position (pixel) in an image (or region of an image), and the shape of each image/representation corresponds to the boundaries within which the nucleus (or nuclear component) lies.

The image analysis process of the present invention analyzes the components of each image/representation (typically on a pixel-by-pixel basis) and derives various parameters to mathematically characterize the nucleus of individual cells. These parameters correspond to biologically significant features such as, for example, the shape, size, concentration, aspect, and amount of nuclear component (e.g., nuclear DNA) of individual cells. The mathematical characterization provides phenotypic information about the cells' nucleus/DNA and can be used to classify cells. Furthermore, from this information, mechanisms of action of drugs, and other important biological information can be deduced.

Feature Extraction and Calculation

Parameters that can be extracted from each image/representation include, but are not limited to, the maximum of grey levels, minimum of grey levels, average of grey levels, and standard deviation of grey levels, length of nucleus major axis, length of nucleus minor axis, nucleus elongation (i.e., ration of major axis/minor axis), nucleus area (i.e., size of nucleus), nucleus perimeter, nucleus compactness, nucleus convex perimeter (i.e., perimeter of the convex hull), and nucleus roughness (i.e., ration of convex perimeter/perimeter). These parameters can serve as cytological descriptors to quantitatively describe and analyze the cell cycle mechanisms.

Image analysis routines for extracting these various parameters can be designed using well-known principles (see, for example, “The Image Processing Handbook”, J. C. Russ (Ed.), 3^{rd} Edition, 1999, CRC Press/LCC IEEE Press, which is incorporated herein by reference in its entirety). In addition, various commercially available tools provide suitable extraction routines. Examples of some of these products include the MetaMorph Imaging System, provided by Universal Imaging Corporation (West Chester, Pa.), and the NIH Image, provided by Scion Corporation, (Frederick, Md.).

Since the values of the extracted features have completely different ranges, it may be desirable to perform an objective scaling, for example, by calculating z scores (see, for example, L. Kaufman and P. J. Fousseeuw, “Finding Groups in Data: An Introduction to Cluster Analysis”, Wiley: New York, 1990, which is incorporated herein by reference in its entirety), using the following equation:

$${z}_{ij} = \frac{{x}_{ij} - {\overline{m}}_{j}}{{s}_{j}} \qquad ( 2 )$$

wherein xᵢⱼ represents the j-th feature of the i-th nucleus and \overline{m}ⱼ is the mean value of all n cells for feature j, and sⱼ is the mean absolute deviation, which is determined by:

$${s}_{j} = \frac{1}{n} {\sum }_{i = 1}^{n} \vert {x}_{ij} - {\overline{m}}_{j} \vert \qquad ( 3 )$$

Feature Subset Selection with K-Nearest Neighbor Classifier

Reducing the dimensionality of a problem is often an important step before any data analysis can be performed. In particular, reducing the dimension by eliminating irrelevant and/or redundant features while preserving most of the information contained in the original data according to some optimality criteria generally provides a better classification accuracy due to finite sample size effects (A. K. Jain and B. Chandrasekaran “Dimensionality and sample size considerations”, In: “Pattern Recognition Practice”, P. R. Krishnaiah and L. N. Kanal (Eds), 1982, Vol. 2, Ch. 39, pp. 835-855, which is incorporated herein by reference in its entirety).

Accordingly, in certain embodiments of methods of the invention, cell phase classification processes comprise a step of feature selection. One goal of feature selection is to choose a subset of features from the set of extracted features that are the most relevant for discrimination and that minimize classification error rate. Using an exhaustive search to determine the optimal feature set is generally infeasible due to the large amount of testing that would be involved.

Various feature selection algorithms and pattern recognition techniques are known and can be used in the practice of the present invention to identify cell-cycle phases based on features extracted from nuclei images. Feature selection methods can be broadly categorized into two groups: the wrapper model and the filter model. Filter methods use feature selection as a preprocessing step to classification while wrapper methods use classification internally as a means of selecting features (see, for example, “Pattern Recognition Practice”, P. R. Krishnaiah and L. N. Kanal (Eds), 1982, which is incorporated herein by reference in its entirety).

In certain embodiments, an optimal feature set is determined by using a sequential forward selection (SFS) method (as described, for example, in J. Kittler, In: “Feature Selection Algorithm, Pattern Recognition and Signal Processing”, Sijthoff & Noordhoof: Germany, 1978, pp. 41-60, which is incorporated herein by reference in its entirety), wherein the discrimination power of the features is evaluated by a K-Nearest Neighbor (KNN) classifier (as described, for example, in A. K. Jain et al., IEEE Trans. on Pattern Analysis and Machine Intelligence, 2000, 20: 4-37, which is incorporated herein by reference in its entirety).

The sequential forward selection (SFS) method is a bottom-up search procedure where features are added one by one to a current (selected) feature subset. At each stage, only one feature is selected from the remaining features and added to the feature subset. The one feature that is selected is that which yields a better classification error rate than any other single remaining feature. The optimal feature subset is found when adding any new feature to the current (selected) feature subset leads to a reduction in the classification error rate.

A K-Nearest Neighbor (KNN) classifier is generally preferred due to its simplicity and flexibility. One goal of a KNN classifier is to provide a criterion to evaluate the discrimination power of the features for feature subset selection. In such a classifier, each cell nucleus is represented as a vector in a p-dimension feature space. The distance d_{B}(x,y) between a cell nucleus x (x=(xₗ,Λ,xₚ)^{t}) and a cell nucleus y (y=(yₗ,Λ,yₚ)^{t}) is defined by the Euclidian distance. A training set T is used to determine the class of a previously unseen nucleus. First, the classifier calculates the distances between an unseen nucleus and all nuclei in the training set. Next, the classifier selects the K cell nuclei in the training set which are the closest to cell nucleus x, and the cell cycle phase of the cell containing nucleus x is determined to be the most common cell cycle phase in the K nearest neighbors.

Correcting Cell Phase Identification Errors

In order to correct for cell phase identification errors and improve the classifier performance, the present invention provides a process wherein biological knowledge-driven heuristic rules are applied during tracking.

In this process, the three following biological rules are used: the phase progression rule, the phase continuation rule, and the phase timing rule. The phase progression rule states that once a cell enters a defined cell-cycle phase, it cannot go back to its previous phase (in other words, it passes a point of no return). The phase continuation rule states that cells cannot skip one phase and enter the phase following the one it skipped. In some cases, a cell may stay in prophase for less than 15 minutes, and this may result in missing a phase in a cell sequence if the temporal resolution used is more than 15 minutes. However, cells cannot jump from metaphase to interphase or from anaphase to metaphase. The phase timing rule states that the time period that a cell stays in a phase also follows certain general rules. According to biological knowledge, the time that a cell stays in interphase is usually more than 20 hours and is generally much longer than the time it stays in various mitotic phases. Cells will usually stay in prophase for no more than 45 minutes; in metaphase for around 1 hour in untreated sequences; and in anaphase for under 1 hour. In time-lapse sequences of drug-treated cell populations, certain cells can stay in metaphase for a longer time or can remain at this phase until the end of the sequence (i.e., arrested metaphase stage).

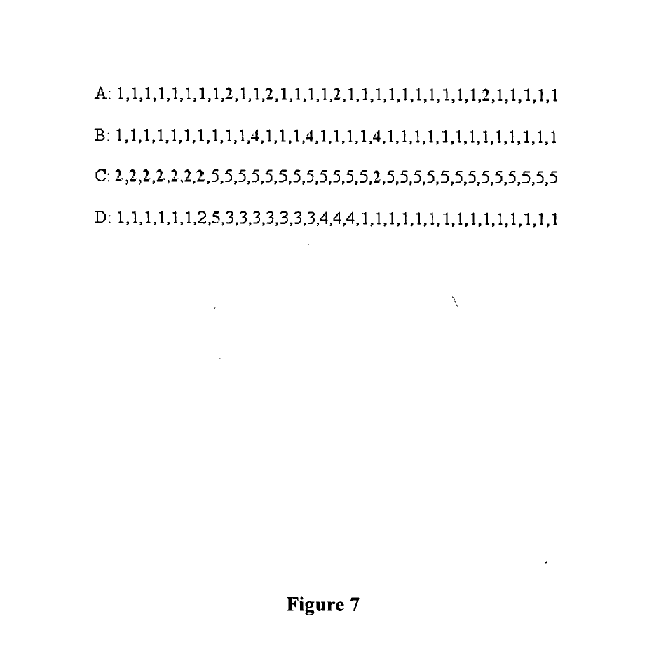

FIG. 7 presents four portions of nucleus sequences containing one or more cell cycle phase identification errors, which are used below to illustrate how to apply the knowledge-driven heuristic rules described above. In these sequences, bold font designates where the errors happened; 1 stands for interphase, 2 for prophase, 3 for metaphase, 4 for anaphase, and 5 for arrested metaphase.

In Case A, the interphase cell is misclassified as prophase cell four times, and these errors can be detected and corrected by applying the phase progression rule.

In Case B, the interphase cell is misclassified as anaphase cell three times, and these errors can be detected and corrected by applying the phase continuation rule. Cells in certain periods of prophase look similar to cells arrested in metaphase. This may cause misclassification between prophase cells and arrested metaphase cells. From a biological point of view, prophase begins when cells start to align their chromosomes and ends when the chromosomes are aligned. When this alignment process cannot be finished because of the influence of drugs, cells are arrested. Thus, cells arrested in metaphase are essentially the same as cells in the middle of prophase. To deal with these kinds of errors metaphase is further divided into normal metaphase and arrested metaphase.

In the first seven frames of Case C, the metaphase cell is misclassified as prophase cell, and these errors can also be detected and corrected by applying the phase timing rule. The remaining one error can be detected and corrected by applying the phase continuation rule.

In Case D, one prophase cell is misclassified as arrested metaphase cell. This error can be corrected by applying the phase timing rule.

Example 2 describes the results obtained using a classification process according to the present invention in time-lapse microscopy images of live GFP-modified HeLa cells. In this example, cell phase identification was performed using a 6-NN (Nearest Neighbor) classifier and a feature subset containing seven features selected from the twelve features extracted from the nuclei images. The experimental results show that the classifier correctly identified nearly all (99%) interphase cells. For cells in metaphase and anaphase, the accuracy of the classifier algorithm was about 83% in each case. However, only 51% of cells in prophase were correctly identified. The classifier made a number of mistakes on separate metaphase cells and prophase cells: 40.4% of prophase cells were wrongly identified as metaphase cells, and 13.1% of metaphase cells were wrongly identified as prophase cells. After application of the knowledge-driven heuristic rules during tracking according to the inventive method, most of the phase identification errors between prophase and metaphase cells were corrected, and the following correct identification rates were obtained: 99.8% for interphase cells; 83% for prophase cells; 95.5% for metaphase cells, and 95.7% for anaphase cells.

C—Tracking

In another aspect, the present invention provides image analysis processes that allow for improved tracking of single cell components in time-lapse, live-cell microscopy image sequences. In particular, using the inventive process it is possible to track cell nuclei even during cell mitosis and division.

The basic principle of single particle tracking is to find for each object in a given time frame its corresponding object in the next time frame. The correspondence is generally based on object features, nearest neighbor information, or other inter-object relationships.

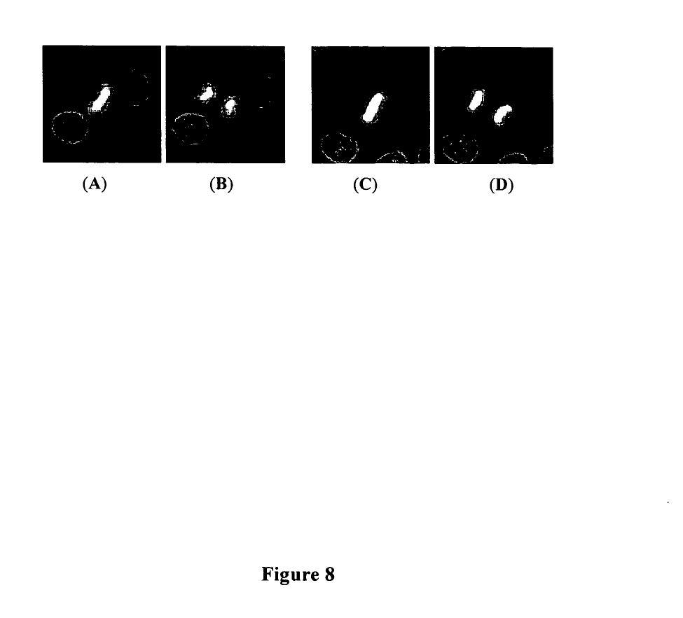

Active contour techniques (M. Kass et al., Int. J. Computer Version, 1988, 1: 321-331) have been used for cell tracking in video-microscopy (see, for example, C. Zimmer et al., IEEE Trans. on Medical Imaging, 2002, 21: 1212-1221; K. A. Giuliano et al., In: “Motion Analysis of Living Cells”, D. R. Soll and D. Wessels (Eds), Wiley: New York, 1998, pp. 53-65; A. P. Goobic et al., Conference Record of the Asilomar Conference on Signals, Systems and Computers, 2001, 1: 88-92; Meas-Yedid and J. C. Olivo-Marin, Proc. IEEE Int. Conf. Image Processing, 2000, 1: 196-199; R. Nilanjan et al., IEEE Trans. on Medical Imaging, 2002, 21: 1222-1235). 100% tracking accuracy has been reported in some of these studies (C. Zimmer et al., IEEE Trans. on Medical Imaging, 2002, 21: 1212-1221; A. P. Goobic et al., Conference Record of the Asilomar Conference on Signals, Systems and Computers, 2001, 1: 88-92; R. Nilanjan et al., IEEE Trans. on Medical Imaging, 2002, 21: 1222-1235). However, active contour trackers require cells to be at least partially overlapped in different frames for successful tracking. In experiments, such as those reported in the Examples of the present invention, where images are captured in a time interval of more than 10 minutes, dividing nuclei can move far away from each other in that time period, and daughter cell nuclei may not overlap with their parents. FIG. 8 shows several examples of nuclei migrating while dividing.

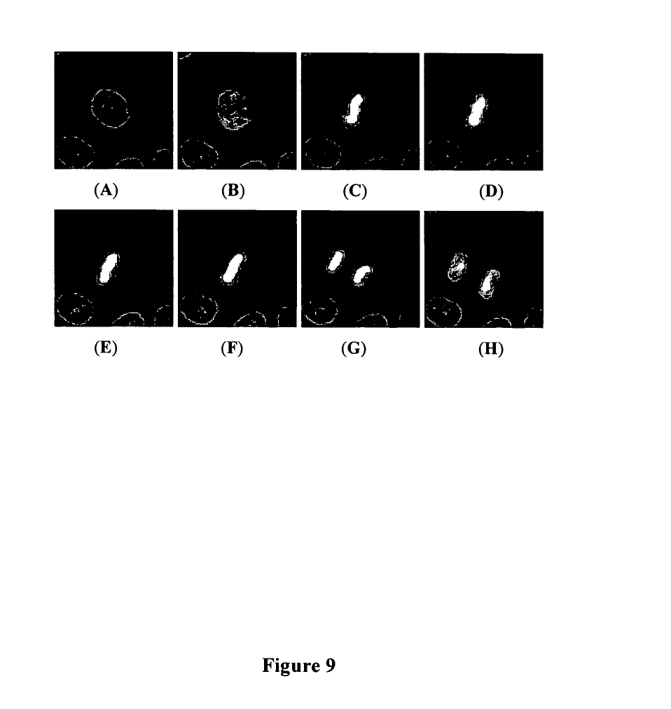

Wang and coworkers (Z. Wang et al., Proc. IEEE International Conference on Systems, Man and Cybernetics, 2000, 3: 1592-1597) have used a Bayesian-based technique for cell tracking by matching several features between cells in consecutive frames and have applied this method in a cell-operation robot vision system. Experiments reported in this paper showed that only 12 out of 20 cells could be successfully tracked in a 48-hour frame cell sequence. This tracking technique is based on the similarity of tracking objects in consecutive frames and fails when a tracking object undergoes dramatic shape and size changes, which is the case of a nucleus undergoing mitosis (as shown in FIG. 9).

Nonlinear shape and size changes means that a correlation tracker (such as that described in A. P. Goobic et al., Conference Record of the Asilomar Conference on Signals, Systems and Computers, 2001, 1: 88-92) will also fail. Some of the difficulties of tracking objects undergoing dramatic shape and size changes can be overcome by using techniques developed for video object tracking (see, for example, S.-F. Chang et al., IEEE Trans. on Circuits and Systems for Video Technology, 1998, 8: 602-615; H. Wang and S.-F. Chang, IEEE Trans. on Circuits and Systems for Video Technology, 1997, 7: 615-628; D. Zhong and S.-F. Chang, IEEE Trans on Circuits and Systems for Video Technology, 1999, 9: 1259-1268). However, all the video object tracking processes, except for the active contour method, are one-to-one tracking techniques, which means that they cannot be used when nuclei are dividing.

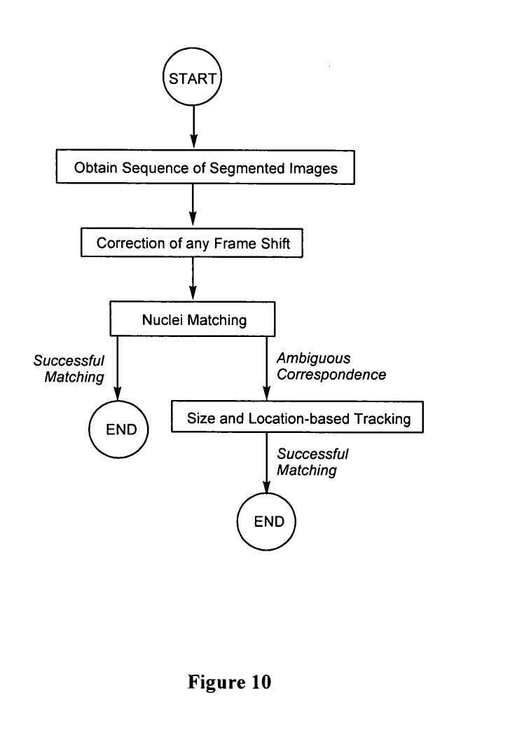

The present invention provides a tracking method that does not suffer from the limitation of existing methods. FIG. 10 shows a high level process flow diagram in accordance with one embodiment of the inventive tracking method. Before describing the different steps of this process, the changes in size and location undergone by a nucleus through the different phases of cell cycle will first be reviewed.

Nucleus Growth, Migration and Division

After division, a cell nucleus will first replicate its chromosome and then grow slowly during the interphase. No drastic size increase takes place. A nucleus grows to its biggest size just before mitosis. After mitosis happens, the size of the nucleus keeps decreasing until it divides. Then another cycle of growth starts.

Nucleus migration in well-sampled time-lapse fluorescence microscopy is usually minute. For example, over a fifteen minute time frame, which is the sampling time period used in all the Examples reported herein, most of the nuclei moved only 5 or fewer pixels, a distance that is much smaller than the size of nuclei. Thus, most cell nuclei will remain in their nearby locations after a lapse of 15 minutes. In addition, nuclei tend to move towards one another. Sometimes two separated nuclei can move so close as to become indistinguishable. In such cases, the segmentation module may not separate them. However, this joint entity will not rotate. When later in the sequence the two nuclei move away from each other, their relative location will not change compared with their relative location before they moved toward each other. In rare cases, small nuclei can partially overlap with nearby large interphase nuclei so that the segmentation module cannot be used to separate them. In such cases, when the small nucleus moves away from the big interphase nucleus, the small nucleus can be recognized by comparing its size to that of the interphase nucleus.

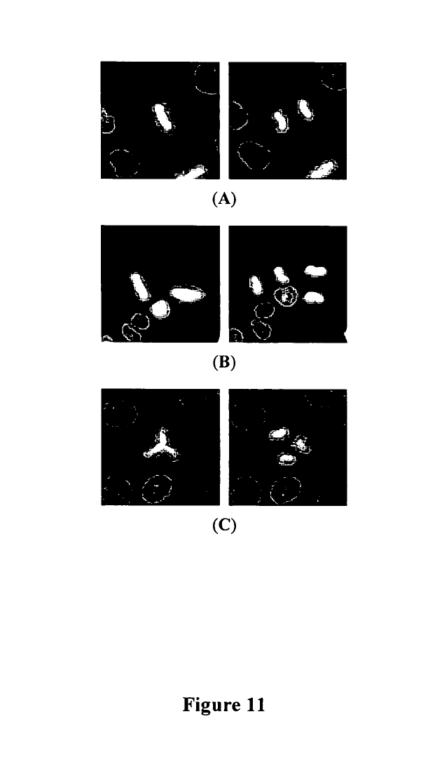

Nucleus migration during division is large considering the small size of metaphase and anaphase nuclei. FIG. 11 shows three different examples of nucleus divisions. Daughter cell nuclei are pulled away from each other by spindles. Since the spindles are located on the opposite sides of the cell body, the daughter cell nuclei will also appear on the opposite sides of their parent location. Thus, the center of gravity of these daughter cell nuclei will remain close to the center of gravity of their parent nuclei.

The size and location-based tracking process of the present invention makes use of all these biological/physiological considerations.

Correction of Frame Shift

The first step in the tracking process of the present invention is aimed at correcting any frame shift before tracking. In a multi-well automated microscopy system, for example, the multi-well plate or microplate is moved back and forth under a CCD camera where pictures are taken of each well. After a well has been imaged, another well is moved under the camera and a new picture is taken. When a previously imaged well is moved back under the camera, the field of view of the second picture may not be exactly the same as the field of view of the first picture. This generally causes a small, in plan shift. By computing a correlation between two images taken, one can find this shift, and use it to correct any relocation problems caused by multi-well plate movements. After correction of any frame shift, nucleus tracking can then be performed.

Nuclei Matching

To simplify the description of the inventive tracking technique, nuclei in two consecutive frames recorded at time t and time t+1 are considered. After nucleus segmentation and correction of any frame shift, a matching process is used to find possible matching nuclei at time t+1 for each nucleus at time t by computing the distance between them. The matching process of the present invention comprises the use of an association matrix to measure these distances. The association matrix is defined as follows:

$$distance = \{ \begin{matrix}1 - \frac{{C}_{surface} ⋂ {Ω}_{surface}}{{C}_{surface} ⋃ {Ω}_{surface}} & {C}_{surface} ⋂ {Ω}_{surface} \neq 0 \\ 1 + \frac{D ( C , Ω )}{Max ( {C}_{size} , {Ω}_{size} )} & {C}_{surface} ⋂ {Ω}_{surface} = 0\end{matrix} \qquad ( 4 )$$

wherein C stands for a nucleus at time t, Ω stands for one of the nuclei appearing in its nearby location at time t+1, D(C,Ω) is the Euclidian distance from the center of gravity of C to the center of gravity of Ω.

The association matrix finds possible matches for nuclei at time t. A match is found if the distance is below a certain threshold. The threshold value is chosen taking into account the fact that when nuclei divide, daughter cell nuclei may not overlap with their parents, which results in a large distance between the parent and daughter nuclei. Therefore, a high threshold value is preferably chosen so that the matching process can find all daughter cell nuclei of a parent nucleus during division. For example, using a small nucleus size of 10 pixels and a maximum nucleus migration distance of 25 pixels, the Applicants have determined and used a threshold value of 3.5.

In the matching process, four different cases can occur: (a) only one nucleus at time t+1 matches a nucleus at time t; (b) no nucleus at t+1 matches a nucleus at time t; (c) a nucleus at time t matches more than one nucleus at time t+1; and (d) more than one nucleus at time t match a nucleus at time t+1. In case (a), a successful match has been found. Case (b) occurs when the nucleus either moves out of the field of view or becomes too dim to be detected. Only the nuclei located at the border of the frame can move out of view. Thus, this situation can be identified simply by checking the nucleus position. Incomplete tracking caused by nuclei moving out of the field of view is not counted in the final tracking statistic. In rare occasions, a nucleus becomes too deemed to be detected, which will generate a tracking error. Case (c) indicates a nucleus split, while case (d) indicates unsuccessful segmentation where two or more nuclei touch or overlap each other and cannot be separated by the segmentation module. Case (c) and case (d) cause ambiguous correspondences between the nuclei at time t and time t+1. These ambiguous correspondences are handled and resolved in the next step of the inventive tracking method.

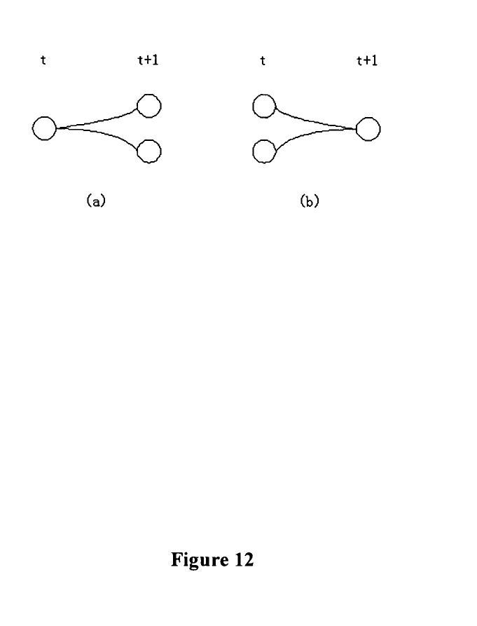

FIG. 12 illustrates the two types of ambiguous correspondences: a one-to-many correspondence and a many-to-one correspondence.

Solving Ambiguous Correspondence

Among the ambiguous correspondences that may be generated by the matching process, some are false ambiguous correspondences. False ambiguous correspondences are due to the large threshold value used in combination with the association matrix (see above) rather than to actual nuclei split or merging. To identify false ambiguous correspondences, a new threshold value is used that takes into account the fact that the change in size undergone by a nucleus that is growing is smaller than the changes in size observed in the case of nuclei splitting, touching or overlapping.

A 10% change threshold is selected to distinguish between the cases in which nuclei size changes are due to actual nuclei growing (changes lower than 10%) and the cases in which nuclei size changes result from unsuccessful segmentation (changes higher than 10%). To identify false ambiguous correspondences, all the ambiguous correspondences identified by the matching process are evaluated using, when possible, the new 10% change threshold.

In a many-to-one correspondence case, the sizes of the nuclei at time t are added to each other one by one according to their distance to the matching nucleus at time t+1. The size of the nucleus located the closest to the matching nucleus is added first, while the size of the nucleus located the furthest from the matching nucleus is added last. Each time the size of a nucleus is added, the sum obtained is compared to the size of the nucleus at time t+1. If the sum is less than 10% larger than the size of the nucleus a time t+1, the size of a new nucleus is added. If the sum is more than 10% larger than the size of the nucleus at t+1, the iterative process is stopped and the last added nucleus is discarded. Only the nuclei added before this last nucleus are considered as matching the nucleus a time t+1.

In a one-to-many correspondence case, simply reversing the selection method used in the many-to-one correspondence case will not work if the correspondence is due to nucleus division as the 10% difference relation does not apply in such a situation (where noticeable nucleus migration takes place). A distinction between the one-to-many correspondence cases caused by nucleus division and the other types of one-to-many correspondence cases can be achieved by checking nuclei size at time t+1 and taking into account the fact that anaphase (i.e., dividing) nuclei have only one chromosome, and thus their size is relatively small compared to the size of nuclei in other phases. For the one-to-many correspondences identified as being caused by other than nucleus division, the 10% change threshold is applied with a reversed selection method. For the one-to-many correspondences identified as being caused by nucleus division, the size of nuclei at time t+1 is compared with a preset threshold value and only the nuclei that have a size lower than the threshold value are considered as resulting from division. For example, in their experiments, the Applicants have used a thresholding value of 250 pixels, as they found that no anaphase nuclei had a size larger than 250 pixels in the time-lapse scans.

After identification of the false ambiguous correspondences, a size and location-based tracking algorithm is used to solve the remaining ambiguous correspondences. This algorithm is based on the following strategy: when nuclei cannot be separated from each other and ambiguous correspondence happens, size and relative location information about these nuclei is recorded and compared with information recorded later in the image sequence when these nuclei move away from each other.

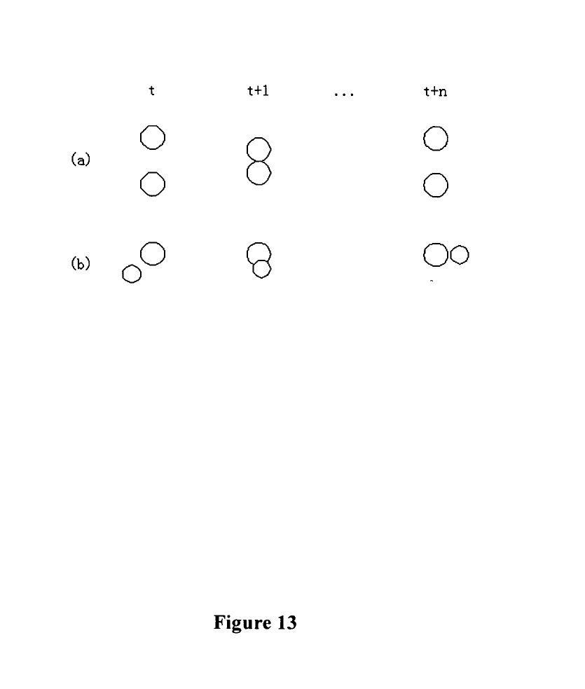

FIG. 13(a) illustrates the way in which size and location-based tracking methods of the invention solve ambiguous correspondence caused by nuclei touching. When two touching nuclei at time t+1 move away from each other at time t+n, their relative location at time t+n is the same as the relative location they had at time t before they moved towards each other. Thus, the relative location information can be used to solve such an ambiguous correspondence. FIG. 13(b) illustrates the way in which size and location based tracking methods of the invention solve ambiguous correspondence caused by nuclei overlapping. When nuclei of different sizes that are touching at time t+1, move away from each other, the correct correspondence can be obtained by associating each nucleus at time t with the one with the same size at time t+n.

One kind of split is caused by over-fragmentation in which a single nucleus is divided into multiple pieces due to incorrect segmentation. This kind of ambiguity can be identified by comparing the change in nucleus size and the relative location of each nucleus at time t+1. If the nuclei at t+1 are separated by one pixel and the sum of their sizes is approximately the same as the size of the nucleus at time t, the fragments at t+1 are merged and considered as a single unit.

Nucleus division can be considered as a special case of nucleus splitting, where one nucleus divides into two or more daughter cell nuclei. If all nuclei at time t+1 only match a single nucleus at time t, these matching nuclei are considered as daughter cell nuclei of the single nucleus. If multiple nuclei divide simultaneously, ambiguous correspondences can happen.

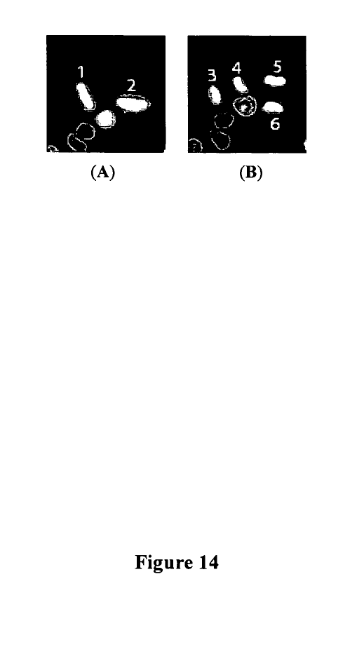

FIG. 14 shows an example of two nuclei dividing simultaneously. In this example, the matching processing finds that daughter cell nucleus 4 matches with both nucleus 1 and nucleus 2; and daughter cell nucleus 5 also matches with both nucleus 1 and nucleus 2.

To solve this ambiguous correspondence the center of gravity of every pair of daughter cell nuclei is calculated. The centers of gravity of nuclei 4 and 3, 4 and 5, and 4 and 6 were first calculated separately. Then the distance from these three centers of gravity to each center of gravity of nucleus 1 and nucleus 2 was calculated. Finally, based on the fact that the distance between the center of gravity for nuclei 4 and 3 and the center of gravity of nucleus 1 is the smallest, nucleus 4 was determined to be the daughter cell nucleus of nucleus 1. Following a similar procedure, nucleus 5 was determined to be the daughter cell nucleus of nucleus 2.

When a touching nuclei entity moves together with another touching nuclei entity, another type of ambiguity takes place. In this case, the size and relative location of each touching entity are recorded, and this information is used together with previous recorded information to solve the ambiguous correspondence when the nuclei cluster divides.

Example 3 describes tracking results obtained in the case of time-lapse microscopy images of live GFP-modified HeLa cells. In these experiments, two tracking methods have been used to track all nuclei contained in four sequences. The first method was the inventive tracking method described above, and the second method was the centroid tracker described by A. P. Goobic (A. P. Goobic et al., Conference Record of the Asilomar Conference on Signals, Systems and Computers, 2001, 1: 88-92). The experimental results show that the inventive tracking method tracked nuclei with an accuracy of 93.4%, which is 5% higher than the centroid tracker. Furthermore, the inventive tracking method was able to keep on tracking nuclei during cell mitosis and division with an accuracy of 94% while the centroid method failed when nuclei touched or overlapped and could not be separated.

### III—Software/Hardware

In general, the image analysis methods of the present invention employ various processes involving data stored in or transferred through one or more computer systems. Accordingly, embodiments of the present invention also relate to an apparatus for performing these operations.

The image analysis processes disclosed herein are not inherently related to any particular computer or other apparatus. Actually, the methods of the present invention may be implemented on various general or specific purpose computing systems. In certain embodiments, the image analysis methods of the present invention may be implemented on a specifically configured personal computer or workstation. In other embodiments, the image analysis methods of the present invention may be implemented on a general-purpose network host machine such as a personal computer or workstation. Alternatively or additionally, the methods of the invention may be, at least partially, implemented on a card for a network device or a general-purpose computing device.

Accordingly, certain embodiments of the present invention relate to computer readable media or computer program products that include program instructions and/or data (including data structures) for performing various computer implemented operations. Examples of computer readable media include, but are not limited to, magnetic media such as hard disks, floppy disks, and magnetic tapes; optical media such as CD-ROM disks; magneto-optical media; semiconductor memory devices, and hardware devices that are specifically configured to store and perform program instructions, such as read-only memory devices (ROM) and random access memory (RAM). The data and program instructions of the present invention may also be embodied on a carrier wave or other transport medium. Examples of program instructions include both machine code, such as produced by a compiler, and files containing higher level code that may be executed by the computer using an interpreter.

### IV—Applications of the Dynamic Cellular Image Analysis Methods

As already mentioned above, time-lapse microscopy has become an important means to study and quantitate the response of individual cells in a cell population to perturbations such as drug treatments. Furthermore, time-lapse, live-cell microscopy can provide far richer information content than conventional fixed-cell microscopy techniques. It also has the potential to make significant contributions to the field of cellular biology by yielding more precise quantitative and multi-parametric characterization of cell cycle mechanisms than existing methods.

By allowing for automated, improved quantitative analysis of dynamic cell images, the processes and apparatus of the present invention will find numerous applications as powerful informatics tools which will help realize the full potential of time-lapse microscopy techniques. In particular, the inventive processes and apparatus will provide a reliable automated solution to process and analyze large volumes of time-lapse microscopy image datasets and to investigate dynamic cellular behaviors.

New Discovery Tools for Novel Anti-Mitotic Drugs and Cell-Line Characterization

The ability to track and quantify cellular components with high spatial and temporal resolution is a key to understanding cell biological processes and to the development of effective therapeutic agents. In particular, the processes and apparatus of the present invention can be used to screen compounds for their ability to affect the cell cycle.

For example, cancer is increasingly viewed as a cell cycle disease. This view reflects the evidence that the vast majority of tumors have suffered defects that derail the cell cycle machinery leading to increased cell proliferation. Such defects can target either components of the cell cycle itself or elements of upstream signaling cascades that eventually converge to trigger cell cycle events.

Existing cancer drug treatments can induce changes in apoptosis and protein localization that are readily detected by time-lapse microscopy. However, whether these changes simply reflect cytotoxicity or actual mechanism of action, or both, has not well be studied. The processes and apparatus of the present invention will enable biomedical scientists and researchers to conduct large scale, systematic studies to measure cell cycle progression, as well as initiation and rate of apoptosis in individual cells as a function of time and in response to a particular drug treatment or combination of drug treatments. These new methods will permit to dissect dynamic cellular processes and to discover the mechanism(s) of action of existing and novel anti-mitotic drugs.

Cancer is not the only clinical condition thought to be associated with cell cycle deregulation (M. D. Garrett, Curr. Sci. 2001, 81: 515-522). Actually, a clear understanding of the mechanism of the cell cycle in the presence or the absence of perturbations can pave the way for new methods for controlling or treating other human diseases, such as certain cardiovascular diseases and certain neurodegenerative diseases.

For example, the mechanism by which neurons die in human neurodegenerative diseases remains an enigma till today (I. Vincent et al., Prog. Cell Cycle Res., 2003, 5: 31-41). Terminally differentiated neurons of normal brains are incapable of cell division. However, accumulating evidence has suggested that aberrant activation of the cell cycle in certain neurodegenerative diseases leads to their demise. Elucidating the details of this cell cycle-mediated degenerative cascade may lead to novel strategies for curbing the onset and progression of certain neurodegenerative diseases.

Similarly, it is known that manipulation of cell division can have beneficial or pathological consequences on cardiovascular function (M. Boehm and E. G. Nabel, Prog. Cell Cycle Res., 2003, 5: 19-30). The inability of cardiomyocytes to proliferate and regenerate following injury results in an impairment of cardiac function associated with physical impediment and may lead to death. The genetic program in the cardiomyocytes that leads to their inability to proliferate and regenerate is not understood, but if identified, it could lead to therapies aimed at re-initiating the cell cycle and proliferation in cardiomyocytes.

Alternatively, the inventive processes and apparatus of image analysis can be used to investigate other biological problems involving dynamic cellular processes. In particular, they can be used for the standardization and characterization of existing cell lines used in drug discovery and biological experiments.

Specific processes, systems and apparatus have generally been used above to describe the present invention. However, it should be understood that the present invention has a much broader range of applicability. In particular, the present invention is not limited to a specific kind of cell component. In cell cycle studies, for example, cellular markers other than the cell nucleus/DNA may be used to measure cell cycle progression. These markers may be any organelle, membrane, molecular structure, molecule that undergoes detectable changes (in shape, size, level of expression, chemical composition, localization and/or distribution within the cell) at one or more stages of the cell cycle. Examples of such cellular markers include centrosomes; histone proteins; cytoskeletal proteins such as actin, vimentin and tubulin; cyclins such as, for example, the mitotic cyclins A and B; and certain members of the kinesin superfamily of microtubule motor proteins. Thus in some embodiments, the processes and apparatus of the present invention can be used to obtain information about one of these cellular markers. In other embodiments, the processes and apparatus of the present invention can be used to obtain information about multiple markers (wherein, optionally, one of the cellular markers is nuclear DNA). One of ordinary skill in the art would recognize other variations, modifications, and alternatives.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Most the results presented in this section have been described by the Applicants in scientific publications (X. Chen et al., “Automated Segmentation, Classification, and Tracking Cell Nuclei in Time-Lapse Microscopy”, IEEE, Trans. Biomedical Engineering, Conference Proceedings, submitted on Jul. 7, 2004; X. Chen et al., “Knowledge-driven cell phase identification in time-lapse microscopy,” IEEE Life Science Data Mining Workshop, Brighton, England, November 2004; and X. Chen et al., “An Automated Method for Cell Phase Identification in High-throughput Time-lapse Screens”, in “Life Science Data Mining”, S. T. C. Wong and C. S. Li (Eds.), World Scientific Inc., accepted for publication). Each of these publications is incorporated herein by reference in its entirety.

### General Information

Four dynamic cellular nucleus sequences were used to test the efficiency of the new method of image analysis disclosed herein. Each sequence consisted of ninety-six frames which were recorded over a period of 24 hours. The sequences were recorded at a spatial resolution of 672×512, and a temporal resolution of one image per 15 minutes using an automated time-lapse fluorescence Nikon TE2000F microscope.

The cells used in the experiments described below were human epithelial cells from the HeLa cell line (cervical carcinoma). Two types of sequences were used to denote drug treated and untreated cells. Some or all of the cells in the treated samples were arrested in metaphase while the cell cycle progress of untreated cell was unaffected. In the absence of drug treatment, HeLa cells usually undergo one division within 24 hours.

A Window-based C/C++ application program was developed by the Applicants to implement the segmentation, classification and tracking algorithms disclosed herein. For an image with approximately 300 nuclei, the average computation time was 1.4 seconds on a Pentium IV 2.4 GHz computer. Only those nuclei entirely contained in the image during the entire sequence were analyzed (these nuclei are hereafter called “target nuclei). Nuclei that left the field of view or appeared in the field of view during the sequence were ignored. The number of target nuclei in each sequence ranged from 78 to 204. After 24 hours, the number of nuclei could grow to more than 400 for untreated sequences.

### Example 1

### Segmentation

To test the segmentation algorithm disclosed herein (i.e., a global thresholding/watershed algorithm combined with shape and size-based merging technique), four images were selected from each cell sequence, generating a test set of 16 images containing a total of 3,071 nuclei. Two other segmentation techniques (namely, a simple watershed algorithm without fragment merging; and the watershed algorithm combined with connectivity-based merging described by Umesh Adiga and Chaudhuri (Pattern Recognition, 2001, 34: 1449-1458)) were also used for comparison purposes.

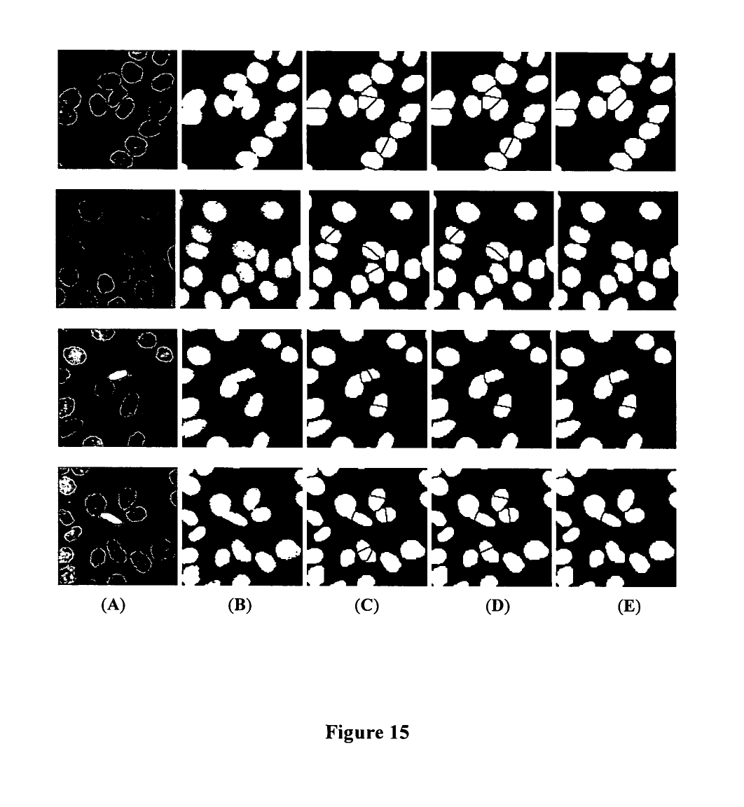

FIG. 15 shows examples of results obtained using these different segmentation techniques. Clearly, the inventive shape and size-based merging method can merge a lot more over-segmented nuclei than the other two methods.

Table 1 presents the segmentation results, which are compared with results obtained by manual analysis. The inventive method correctly segmented 97.8% of the nuclei. The watershed algorithm caused 165 nuclei out of 3,071 nuclei to be over-segmented. The connectivity-based merging technique used by Umesh Adiga and Chaudhuri could only merge 36.4% of them, while the proposed shape and size-based method merged 82.4%. The connectivity-based merging technique failed because it was unable to deal with fragments whose size was larger than the preset value. In such a case, the fragments were considered as individual nuclei. The shape and size-based technique merged 14 of the 2,880 separated nuclei, while the connectivity-based technique merged 20.

TABLE 1
Segmentation results obtained using different techniques

| | No. Nuclei | Correctly | Over- | Under- |
|---|---|---|---|---|
| | Analyzed | Separated | Segmented | Segmented |
| Watershed | 3071 | 2880 | 165 | 26 |
| | | (93.8%) | (5.4%) | (0.8%) |
| Connectivity- | | 2920 | 105 | 46 |
| based | | (95.1%) | (3.4%) | (1.5%) |
| merging | | | | |
| Size and | | 3002 | 29 | 40 |
| shape-based | | (97.8%) | (0.9%) | (1.3%) |
| merging | | | | |

### Example 2

### Cell Phase Identification

The training of the feature selection method was carried out using 100 nuclei for each cell cycle phase which resulted in a training set of 400 nuclei. The 400 cell nuclei were evenly divided into five disjointed subsets. Selection performance was evaluated by a five-fold cross validation in five individual tests with ⅘^{th} of the initial data serving as the training set for the selection algorithm. The remaining ⅕^{th} of the data served as the test set. In exhaustive experiments, a six nearest-neighbor (6-NN) rule delivered the most reliable results for the different selection strategies.

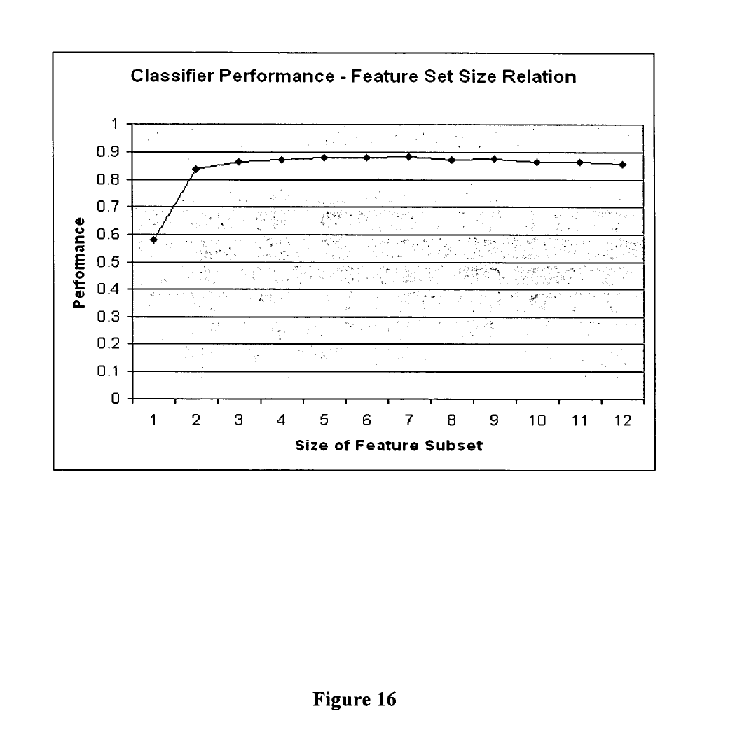

FIG. 16 shows the variation of the performance of the classifier (which is defined as the ratio between the number of nuclei correctly identified and the total number of nuclei) as a function of the size of the feature subset. The best performances were achieved with a subset size of seven features. Addition of the remaining 5 features caused a decrease in the selection percentages. The features in the optional feature set and the order in which they were selected by the SFS method were as follows: Perimeter, Standard Deviation of Grey Levels, Compactness, Maximum Intensity of Grey Levels, Major Axis, Mean of Grey Levels, and Minor Axis. These seven features were then used for cellular phase identification.

A total of 80 nuclei were selected from the four sequences. Each nucleus was tracked for 12.5 hours. Thus 50 images were taken for each nucleus. During this time, these 80 cells either divided or were arrested in metaphase. This process generated a test set with 4,000 nuclei. The cell phase identification experiments were performed using this test set. The cell identification was carried out with a 6-Nearest Neighbor classifier based on the seven derived features. The training set for the classifier consisted of the 400 nuclei used for feature selection. Table 2 presents the experimental results obtained using the inventive method compared to results obtained by manual analysis.

TABLE 2
Cell phase identification results

| | Assigned | | | | | |
|---|---|---|---|---|---|---|
| | Inter- | Pro- | Meta- | Ana- | | |
| True | phase | phase | phase | phase | Unknown | Accuracy |
| Interphase | 2763 | 1 | 22 | 2 | 2 | 99% |
| (2790) | | | | | | |
| Prophase | 3 | 24 | 19 | 1 | 0 | 51.1% |
| (47) | | | | | | |
| Metaphase | 23 | 125 | 792 | 11 | 1 | 83.2% |
| (952) | | | | | | |
| Anaphase | 2 | 0 | 32 | 175 | 0 | 83.7% |
| (209) | | | | | | |

The inventive classifier correctly identified nearly all (99%) interphase cells. For cells in metaphase and anaphase, the accuracy of the classifier algorithm was about 83% for each cell cycle phase. However, only 51.1% of cells in prophase were correctly identified. The classifier made a number of mistakes on separate metaphase cells and prophase cells. 40.4% of prophase cells were wrongly identified as metaphase cells, and 13.1% of metaphase cells were wrongly identified as prophase cells.

Table 3 summarizes the phase identification results obtained by applying the knowledge-driven heuristic rules to the classifier outputs. Note that most of the phase identification errors between prophase and metaphase cells were corrected; and cells were identified as metaphase cells. The phase identification correct rate increased by 0.8% for interphase cells, 31.9% for prophase cells, 12.3% for metaphase cells, and 12% for anaphase cells.

TABLE 3
Cell phase identification results after applying
knowledge-driven heuristic rules

| | Assigned | | | | | |
|---|---|---|---|---|---|---|
| | Inter- | Pro- | Meta- | Ana- | | |
| True | phase | phase | phase | phase | Unknown | Accuracy |
| Interphase | 2785 | 0 | 4 | 1 | 0 | 99.8% |
| (2790) | | | | | | |
| Prophase | 3 | 39 | 4 | 1 | 0 | 83% |
| (47) | | | | | | |
| Metaphase | 7 | 31 | 909 | 5 | 0 | 95.5% |
| (952) | | | | | | |
| Anaphase | 1 | 0 | 8 | 200 | 0 | 95.7% |
| (209) | | | | | | |

### Example 3

### Cell Nuclei Tracking

To establish a metric for the performance of the tracking algorithm, three types of factors have been considered:
(a) percentage of nuclei tracked (which is the number of nuclei tracked without termination through the entire sequence divided by the total number of nuclei at the beginning);
(b) percentage of divisions detected (which is the ratio between the number of cell divisions for which the daughter cell nuclei were correctly assigned to their parent and the total number of cell divisions); and
(c) false division number (which is the number of false divisions detected where two or more nuclei are associated with one nucleus in a previous frame, which did not undergo division).

Two tracking methods have been used to track the nuclei in all of the four sequences described above. The first method used was the location and size-based tracker disclosed herein and the second method was the centroid tracker (A. P. Goobic et al., Conference Record of the Asilomar Conference on Signals, Systems and Computers, 2001, 1: 88-92).

Table 4 shows a comparison of the tracking rates obtained by each tracking method. The inventive tracking method achieved an average 94.3% tracking accurate rate, which is 5% higher than the centroid tracker. The centroid method failed when nuclei touched or overlapped and could not be separated. By using size and location information, the inventive tracking method was able to successfully resolve the ambiguous correspondences caused by nucleus touching and overlapping, and this resulted in an increase in tracking accuracy.

TABLE 4
Tracking performance comparison between different techniques.

| | | Location and Size | | | |
|---|---|---|---|---|---|
| | Nuclei | based Tracker | | Centroid Tracker | |
| Sequences | Number | Tracked | Missed | Tracked | Missed |
| A (untreated) | 204 | 188 | 16 | 184 | 20 |
| B (untreated) | 90 | 82 | 8 | 80 | 10 |
| C (treated) | 133 | 130 | 3 | 118 | 15 |
| D (treated) | 78 | 76 | 2 | 69 | 9 |
| Total | 505 | 476 | 29 | 451 | 54 |
| | | (94.3%) | (5.7%) | (89.3%) | (10.7%) |

Table 5 shows the performance of the inventive method in detecting divisions. The inventive tracking module correctly associated 94% daughter cell nuclei with their parents. Errors happened, most commonly, when daughter cell nuclei overlapped with nearby nuclei right after division. In this case, the segmentation module was not able to separate these daughters from the nuclei under them. False division was mostly caused by over-segmentation. Both situations can be handled by improving the efficiency of the nucleus segmentation module.

TABLE 5
Division detection results.

| Image | No. of | | | | | False |
|---|---|---|---|---|---|---|
| Sequence | Divisions | Detected | | Missed | | Divisions |
| A (untreated) | 62 | 57 | | 5 | | 6 |
| B (untreated) | 57 | 51 | | 6 | | 5 |
| C (treated) | 80 | 79 | | 1 | | 0 |
| D (treated) | 0 | 0 | | 0 | | 3 |
| Total | 199 | 187 | (94%) | 12 | (6%) | 14 |

### Discussion

Time-lapse fluorescence microscopy is becoming an important method to study dynamic cellular processes over a large population of cells, with significant potential in achieving new, high-throughput ways of conducting drug discovery and quantitative cellular studies. The commercial availability of automated, multi-plate platforms in time-lapse microscopes further allows the biologists to conduct a large number of biological experiments in parallel and significantly increases the throughput of data acquisition.

The new method of image analysis disclosed herein allows segmentation, classification, and tracking studies of large volumes of dynamic cellular image data to be performed automatically.

The experimental results obtained using the inventive implemented method show an accuracy of 97.8% on nucleus segmentation. By applying feature selection strategies, the number of features was reduced from 12 to 7. Experiments showed that the classifier achieved the following correct identification performance on the three cell cycle phases: interphase: 99%, metaphase: 83.2%, and anaphase: 83%. Using the biological knowledge-driven heuristic rules, the tracking module corrected most of the prophase identification errors and improved the correct classification rate from 51.1% to 83%. The corrected rates for metaphase and anaphase were also improved to 95.5% and 95.7%, respectively.

Furthermore, the inventive tracking algorithm disclosed herein can also deal successfully with non-linear changes that occur during cell mitosis. In the inventive tracking method, ambiguous correspondences are solved after nuclei move away from each other. By combining these features, the method allows to keep tracking nuclei and to analyze their changes over a longer period of time. The 94.3% tracking rate shows the robustness of the inventive method of analysis.

Active contour techniques have been used to handle division (C. Zimmer et al., IEEE Trans. on Medical Imaging, 2002, 21: 1212-1221). However, these methods cannot track dividing nuclei in time-lapse microscopy. Using the inventive method, the daughter cell nuclei are found and identified by a matching process. A final correspondence between daughter cell nuclei and their parents is obtained by matching the center of gravity of daughter cell nuclei with the centers of gravity of their parents. Experiments show that the inventive tracking method is able to correctly detect 94% of nucleus divisions.

In summary, an automated method of dynamic cellular image analysis was designed and implemented. The method shows high accuracy on both cell phase identification and tracking and is currently being used in high-throughput cancer drug screening studies at the Applicants' institution.

The inventive method is the first technique that can be used to automatically track and identify cell-cycle phases of individual cells in time-lapse microscopy studies. In particular, it is the first method that allows identification of the different mitotic phases to be carried out. The availability of this method will realize the full potential of time-lapse microscopy and greatly increase the productivity of high-content drug screening by eliminating laborious and subjective manual analysis operations.

The next step of this research will be to extract attributes or features from vast volumes of time-lapse images of cancer cell lines under different drug perturbation conditions and create a large cellular imaging database. Data mining and knowledge modeling techniques (X. Zhou et al., J. Franklin Institute-Engineering and Applied Mathematics, 2004, 341: 137-156; X. Zhou et al., IEEE/ACM Trans. on Computational Biology and Bioinformatics, 2004, in press) will then be used to study the influence of various drugs compounds on the mitotic process of cancer cells. This will allow the identification of effective lead candidates of anti-mitotic cancer drug compounds for further evaluation in the Applicants' laboratory of drug development at the Harvard Center for Neurodegeneration and Repair and at the drug discovery laboratory of the Institute of Chemistry and Cell Biology, Harvard Medical School.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for classifying a cell into a cell cycle state, the method comprising steps of:
receiving a cell image showing the nucleus of one or more cells;
performing a segmentation analysis of the cell image to obtain a segmented digital image;
extracting one or more parameters from the segmented digital image to characterize the nucleus of at least one of the cells of the cell image; and
classifying the at least one cell into a cell cycle state based on the one or more extracted parameters.

2. The method of claim 1, wherein the cell cycle state is selected from the group consisting of mitotic, interphase, prophase, metaphase, anaphase, and arrested metaphase.

3. The method of claim 1, wherein performing a segmentation analysis comprises steps of:
performing a global threshold analysis of the cell image to generate a binary image;
applying a watershed algorithm to segment any touching nuclei present in the binary image; and
merging fragments of any over-segmented nuclei generated by the watershed algorithm using a shape and size merging process.

4. The method of claim 3, wherein performing a global threshold analysis comprises using an isodata algorithm.

5. The method of claim 3, wherein the shape and size merging process comprises steps of:
measuring the size, T_{size}, of the smallest nucleus in the cell image;
identifying a first fragment touching a second fragment, wherein the second fragment is the smallest fragment touching the first fragment;
if the size of the first fragment is lower than T_{size}, merging the first and second fragments;
if the size of the first fragment is greater than T_{size}, calculating the compactness of the first fragment, the compactness of the second fragment and the compactness of an object consisting of the first fragment merged with the second fragment; and
if the compactness of the object is lower than the compactness of the first fragment or of the second fragment, merging the first and second fragments.

6. The method of claim 5, wherein the shape and size merging process further comprises repeating steps of the process.

7. The method of claim 1, wherein the nucleus of one or more cells of the cell image is labeled with a detectable agent.

8. The method of claim 7, wherein the detectable agent is associated with a nuclear component.

9. The method of claim 8, wherein the nuclear component is selected from the group consisting of nuclear DNA, nuclear proteins, and combinations thereof.

10. The method of claim 8, wherein the detectable agent produces a signal whose intensity is proportional to the amount of nuclear component with which it is associated.

11. The method of claim 10, wherein the segmented digital image comprises a representation of the nucleus of each of the one or more cells, each representation comprising a collection of signal intensity values at positions in the image where the nuclear component is present.

12. The method of claim 11, wherein the step of extracting one or more parameters from the segmented digital image to characterize the nucleus of at least one cell of the cell image comprises extracting from the representation of each nucleus to be characterized a feature selected from the group consisting of maximum of grey levels, minimum of grey levels, average of grey levels, standard deviation of grey levels, length of nucleus major axis, length of nucleus minor axis, nucleus elongation, nucleus area, nucleus perimeter, nucleus compactness, nucleus convex perimeter, nucleus roughness, and combinations thereof.

13. The method of claim 12, wherein the step of classifying the cell into a cell cycle state based on the one or more parameters comprises selecting a set of extracted features using a classifier.

14. The method of claim 13, wherein selecting a set of extracted features comprises using a Sequential Forward Selection method.

15. The method of claim 13, wherein the classifier is a K-Nearest Neighbor classifier.

16. The method of claim 15, wherein the classifier is optimized with training data.

17. The method of claim 1, wherein the cell image is part of a sequence of cell images recorded at consecutive time points, wherein each cell image is associated with a specific time point.

18. The method of claim 17 further comprising steps of:
performing a segmentation analysis of each cell image of the sequence to obtain a sequence of segmented digital images, wherein each segmented digital image is associated with the time point of the cell image from which it is obtained;
extracting one or more parameters from each segmented digital image to characterize the nucleus of at least one cell at each time point of the sequence; and
classifying the at least one cell into a cell cycle state based on the one or more extracted parameters at each time point of the sequence.

19. The method of claim 18 further comprising tracking the nucleus of the at least one cell over the sequence of images.

20. The method of claim 19, wherein tracking the nucleus of the at least one cell over the sequence comprises steps of:
(a) performing a correction of any frame shift in the segmented digital images;
(b) applying a matching algorithm to find, for each nucleus in a first image of the sequence, possible matching nuclei in a second image of the sequence, wherein the second image is consecutive to the first image; and
(c) repeating step (b).

21. The method of claim 20, wherein applying a matching algorithm to find possible matching nuclei in a second image for each nucleus in a first image comprises steps of:
calculating, for each nucleus in the first image, the distance between the nucleus and a possible matching nucleus in the second image; and
determining that the nucleus in the second image matches the nucleus in the first image if the distance calculated is below a distance threshold D.

22. The method of claim 21, further comprising solving any ambiguous correspondences generated by the matching algorithm.

23. The method of claim 22, wherein solving any ambiguous correspondences comprises steps of:
identifying any false ambiguous correspondences; and
applying a size and location-based tracking algorithm to solve the remaining ambiguous correspondences.

24. The method of claim 23, wherein applying the size and location-based tracking algorithm comprises calculating one or more of nucleus size, nucleus size change from one image to another, nucleus location, nucleus location change from one image to another, relative size of two nuclei in an image, relative location of two nuclei in an image, relative size change of two nuclei from one image to another, relative location change of two nuclei from one image to another, nucleus center of gravity, distance between two centers of gravity, and combinations thereof.

25. The method of claim 19, further comprising correcting any classification errors.

26. The method of claim 25, wherein correcting any classification errors comprises applying knowledge-driven heuristic rules.

27. The method of claim 1, wherein the one or more cells are primary cells, secondary cells or immortalized cells.

28. The method of claim 27, wherein the one or more cells are mammalian cells.

29. The method of claim 28, wherein thee one or more cells are human cells.

30. The method of claim 28, wherein the one or more cells comprise cells treated under control conditions.

31. The method of claim 28, wherein the one or more cells comprise cells treated with a test agent.

32. The method of claim 28, wherein the one or more cells are in a multi-well assay plate.

33. A machine readable medium on which are provided program instructions for classifying a cell into a cell cycle state, the program instructions comprising:
program code for receiving a cell image showing the nucleus of one or more cells;
program code for performing a segmentation analysis of the cell image to obtain a segmented digital image;
program code for extracting one or more parameters from the segmented digital image to characterize the nucleus of at least one of the cells of the cell image; and
program code for classifying the at least one cell in to a cell cycle state based on the one or more extracted parameters.

34. The machine readable medium of claim 33, wherein the cell cycle state is selected from the group consisting of mitotic, interphase, prophase, metaphase, anaphase, and arrested metaphase.

35. The machine readable medium of claim 33, wherein program code for performing a segmentation analysis comprises:
program code for performing a global threshold analysis of the cell image to generate a binary image;
program code for applying a watershed algorithm to segment any touching nuclei present in the binary image; and
program code for merging fragments of any over-segmented nuclei generated by the watershed algorithm using a shape and size merging process.

36. The machine readable medium of claim 35, wherein program code for performing a global threshold analysis comprises program code for using an isodata algorithm.

37. The machine readable medium of claim 35, wherein program code for merging fragments of any over-segmented nuclei using a shape and size merging process comprises:
program code for measuring the size, T_{size}, of the smallest nucleus in the cell image;
program code for identifying a first fragment touching a second fragment, wherein the second fragment is the smallest fragment touching the first fragment;
program code for merging the first and second fragments if the size of the first fragment is lower than T_{size};
program code for calculating the compactness of the first fragment, the compactness of the second fragment, and the compactness of an object consisting of the first fragment merged with the second fragment if the size of the first fragment is greater than T_{size}; and
program code for merging the first and second fragments if the compactness of the object is lower than the compactness of the first fragment or of the second fragment.

38. The machine readable medium of claim 37, wherein the nucleus of one or more cells of the cell image is labeled with a detectable agent.

39. The machine readable medium of claim 38, wherein the detectable agent is associated with a nuclear component.

40. The machine readable medium of claim 39, wherein the nuclear component is selected from the group consisting of nuclear DNA, nuclear proteins, and combinations thereof.

41. The machine readable medium of claim 39, wherein the detectable agent produces a signal whose intensity is proportional to the amount of nuclear component with which it is associated.

42. The machine readable medium of claim 41, wherein the segmented digital image comprises a representation of the nucleus of each of the one or more cells, each representation comprising a collection of signal intensity values at positions in the cell image where the nuclear component is present.

43. The machine readable medium of claim 42, wherein program code for extracting one or more parameters from the segmented digital image to characterize the nucleus of at least one of the cells of the cell image comprises program code for extracting from the representation of each nucleus to be characterized a feature selected from the group consisting of maximum of grey levels, minimum of grey levels, average of grey levels, standard deviation of grey levels, length of nucleus major axis, length of nucleus minor axis, nucleus elongation, nucleus area, nucleus perimeter, nucleus compactness, nucleus convex perimeter, nucleus roughness, and combinations thereof.

44. The machine readable medium of claim 43, wherein program code for classifying the at least one of the cells into a cell cycle state comprises: program code for selecting a set of extracted features.

45. The machine readable medium of claim 44, wherein program code for selecting a set of extracted features comprises: program code for performing a Sequential Forward Selection using a K-Nearest Neighbor classifier.

46. The machine readable medium of claim 45, wherein the classifier is optimized with training data.

47. The machine readable medium of claim 33, wherein the cell image is part of a sequence of cell images recorded at consecutive time points, wherein each cell image is associated with a specific time point.

48. The machine readable medium of claim 47, wherein program instructions further comprise:
program code for performing a segmentation analysis of each cell image of the sequence to obtain a sequence of segmented digital images, wherein each segmented digital image is associated with the time point of the cell image from which it is obtained;
program code for extracting one or more parameters from each segmented digital image to characterize the nucleus of at least one of the cells at each time point of the sequence; and
program code for classifying the at least one of the cells into a cell cycle state based on the one or more extracted parameters at each time point of the sequence.

49. The machine readable medium of claim 48, wherein program instructions further comprise program code for tracking the nucleus of the at least one cell over the sequence of images.

50. The machine readable medium of claim 49, wherein program code for tracking the nucleus of the at least one cell over the sequence comprises:
(a) program code for performing a correction of any image frame shift in the segmented digital images;
(b) program code for applying a matching algorithm to find, for each nucleus in a first image of the sequence, possible matching nuclei in a second image of the sequence, wherein the second image is consecutive to the first image; and
(c) program code for repeating step (b).

51. The machine readable medium of claim 50, wherein program code for applying a matching algorithm to find possible matching nuclei in a second image for each nucleus in a first image comprises:
program code for calculating, for each nucleus in the first image, the distance between the nucleus and a possible matching nucleus in the second image; and
program code for determining that the nucleus in the second image matches the nucleus in the first image if the distance calculated is below a distance threshold D.

52. The machine readable medium of claim 51, wherein program code for applying a matching algorithm further comprises program code for solving any ambiguous correspondences generated by the matching algorithm.

53. The machine readable medium of claim 52, wherein program code for solving any ambiguous correspondences comprises:
program code for identifying any false ambiguous correspondences; and
program code for applying a size and location-based tracking algorithm to solve the remaining ambiguous correspondences.

54. The machine readable medium of claim 53, wherein program code for applying the size and location-based tracking algorithm comprises program code for calculating one or more of nucleus size, nucleus size change from one image to another, nucleus location, nucleus location change from one image to another, relative size of two nuclei in an image, relative location of two nuclei in an image, relative size change of two nuclei from one image to another, relative location change of two nuclei from one image to another, nucleus center of gravity, distance between centers of gravity, and combinations thereof.

55. A computer program product comprising a machine readable medium of claim 33.

56. An image analysis apparatus for classifying a cell into a cell cycle state, the apparatus comprising:
a memory adapted to store, at least temporarily, one or more cell images showing the nucleus of one or more cells; and
a processor configured or designed to classify at least one cell shown on the one or more cell images by performing the method of claim 1.

57. The image analysis apparatus of claim 56 further comprising an interface adapted to receive the cell image.

58. The image analysis apparatus of claim 56 further comprising an image acquisition system that produces the image.
